(19) United States
(12) Patent Application Publication
(10) Pub. No.: US 2021/0270827 A1
(43) Pub. Date: Sep. 2, 2021

(54) CAPILLARY ACTION TEST USING PHOTOLUMINESCENT INORGANIC NANOPARTICLES

(71) Applicants: ECOLE POLYTECHNIQUE, Palaiseau (FR); CENTRE NATIONAL DE LA RECHERCHE SCIENTIFIQUE, Paris Cedex 16 (FR)

(72) Inventors: Pascal PREIRA, Savigny-Sur-Orge (FR); Maximilian RICHLY, Paris (FR); Cédric BOUZIGUES, Paris (FR); Antigoni ALEXANDROU, Palaiseau (FR); Thierry GACOIN, Bures Sur Yvette (FR)

(73) Assignees: ECOLE POLYTECHNIQUE, Palaiseau (FR); CENTRE NATIONAL DE LA RECHERCHE SCIENTIFIQUE, Paris Cedex 16 (FR)

(21) Appl. No.: 17/260,533

(22) PCT Filed: Jul. 17, 2019
(86) PCT No.: PCT/EP2019/069263

§ 371 Date: Jan. 14, 2021

(30) Foreign Application Priority Data
Jul. 18, 2018 (FR) 1856651

(51) Int. Cl. G01N 33/543; C09K 11/77; G01N 33/558; G01N 21/64

(52) U.S. Cl. CPC G01N 33/54346; C09K 11/7794; G01N 33/558; G01N 2458/40; C09K 11/7776; C09K 11/7795; G01N 21/6428; G01N 33/54386

(57) ABSTRACT

The present invention relates to an in vitro method for detecting and/or quantifying a biological or chemical substance of interest in a liquid sample, by a capillary action test using, as probes, photoluminescent inorganic nanoparticles, of formula A₁₋ₓLnₓVO_{4(1-y)}(PO₄)_{y} (II), in which Ln is selected from europium (Eu), dysprosium (Dy), samarium (Sm), neodymium (Nd), erbium (Er), ytterbium (Yb), thulium (Tm), praseodymium (Pr), holmium (Ho) and mixtures thereof; A is selected from yttrium (Y), gadolinium (Gd), lanthanum (La), lutetium (Lu), and mixtures thereof; 0<x<1; and 0≤y<1, said method employing detection of the luminescence, with an emission lifetime shorter than 100 ms, of the nanoparticles, after one-photon absorption, by excitation of the matrix at a wavelength less than or equal to 320 nm.

It also relates to a capillary action test device comprising, as probes, the aforementioned nanoparticles, as well as the use of such a method for purposes of in vitro diagnostics.

The present invention relates to the field of bioanalysis and in vitro diagnostics. It relates more particularly to an in vitro method for detecting and/or quantifying a biological or chemical substance of interest, for example proteins, antibodies, toxins and other compounds, in a liquid sample, by a capillary action test using, as probes, photoluminescent inorganic nanoparticles with controlled optical and physicochemical properties.

Capillary action tests, such as for example lateral flow assays (LFAs), generally known by the name “strip tests”, are commonly used for the purposes of clinical, pharmaceutical, food or chemical analyses. They may be used for detecting the presence of many types of analytes such as antibodies, antigens, proteins, biomarkers, chemical molecules, nucleic acids, etc. ([1]). When the recognition molecules used in the lateral flow assay are antibodies, it is more commonly called an “immunochromatographic assay” (lateral flow immunoassay, LFIA).

The capillary action tests are particularly valued for their simplicity of use, their speed (detection in a time less than or equal to 15 minutes) and their low cost.

In general, devices for capillary action tests employ a means for capillary action in the form of a porous solid support (for example a nitrocellulose membrane), within which the test sample, deposited at one end of the solid support, and the reagents, incorporated in the device as sold, migrate by capillary action.

Typically, the porous solid support of the devices for capillary action tests comprises a labeling zone (“Conjugate Pad” in English-language terminology) bearing, in liquid form, lyophilized or dehydrated, a reagent specifically binding the substance to be analyzed (or “analyte”), conjugated with a probe (or detecting species), and a detection zone (“Detection Pad” in English-language terminology) on which a reagent specifically capturing the analyte is immobilized.

The reagent specifically binding the analyte is immobilized in lyophilized form but becomes mobile in the solid support when wet. Thus, when the solid support is brought into contact with a liquid sample, the latter migrates by capillary action in said support entraining the reagent specifically binding the analyte conjugated to the probe. The sample and the reagent specifically binding the analyte migrate by capillary action in the solid support as far as the detection zone bearing an immobilized capturing reagent, specific to the analyte.

In the commonest capillary action tests, of the “sandwich” type, the binding reagent conjugated to the probe binds to the analyte contained in the sample, when the latter meet, and then the analyte is immobilized on the solid support by the capturing reagent. The presence or absence of the analyte in the sample is thus measured by detecting the probe immobilized at the level of the detection zone via the analyte.

These devices also comprise a so-called control zone, located downstream of the detection zone relative to the direction of capillary flow, in which a second capturing reagent specific to the labeled targeting reagent is immobilized. After migrating as far as the detection zone, the binding reagent coupled to the probe in excess, which has not reacted with the analyte, migrates as far as the control zone, and binds to the second capturing reagent. The user thus has a positive control allowing the migration of the sample and the reagents in the device to be verified, and therefore verifying proper operation of the test.

Determination of the analyte in the sample is therefore achieved by detecting the presence or absence of the probe at the level of the detection zone and, optionally, at the level of the control zone.

The probes most commonly used in capillary action tests are gold nanoparticles ([1], [2]). These absorb light at characteristic wavelengths that correspond to their surface plasmon frequency. The surface plasmon frequency of the nanoparticles depends on their size and their state of aggregation. When gold nanoparticles are immobilized at the level of the detection zone and/or control zone, the absorption associated with the frequency of the surface plasmon of the nanoparticles, which are close together, thus allows a characteristic color, typically blue/violet, visible to the naked eye, to be imparted to said zones. Advantageously, these tests allow the result of analysis of the sample to be obtained quickly, typically in some minutes, compared to the time required for conventional immuno-detection techniques, such as an enzyme-linked immunosorbent assay (ELISA), typically of several hours for the ELISA assays. Moreover, they do not require expensive and bulky equipment for preparation or analysis.

However, these tests have the major drawback of having a low detection sensitivity and a poor limit of quantitation. Therefore they generally provide only qualitative, or semiquantitative, information. In particular, these strip tests have a far lower sensitivity of detection than that obtainable by conventional immuno-detection assays, for example of the ELISA type. Thus, strip tests based on gold nanoparticles typically make it possible to detect concentrations of the order of some ng/mL, whereas an ELISA assay allows detection of some pg/mL, typically 2 to 3 orders of magnitude more sensitive. For example, the company Cortez Diagnostics offers a strip test for detecting troponin I (biomarker of myocardial infarction) having a sensitivity of 1 ng/ml ([3]), whereas the company Abcam offers an ELISA assay (ab200016) with a sensitivity of 7 pg/mL.

In order to improve the sensitivity of strip tests, in other words to lower the limit of detection of these assays, various materials have been proposed as probes as alternatives to gold nanoparticles in capillary action tests, and in particular modified gold nanoparticles, magnetic particles, semiconductor nanocrystals or “quantum dots”, up-conversion phosphors, organic fluorophors, etc. ([1], [4]).

Thus, several nanomaterials based on gold nanoparticles have been explored as probes for devices for capillary action tests, for example such as magnetic microspheres comprising a core formed from a nanometric particle of Fe₂O₃ coated with gold nanoparticles ([5]), silica nanotubes bearing gold nanoparticles ([6]), or multi-branched gold nanoflowers (GNFs) ([7]). We may also cite Der-Jiang et al. ([8]), who propose depositing a layer of silver on gold nanoparticles. However, these approaches require routes for synthesis of these probes that are often extremely complex. Furthermore, as silver oxidizes more easily than gold, probes combining silver with gold are less stable for application in a capillary action test in an aqueous medium.

The use of photoluminescent probes (also called more simply “luminescent probes” hereinafter), such as organic fluorophors and quantum dots, has made it possible to increase the sensitivity of the capillary action tests, compared to assays based on gold nanoparticles. In fact, detection of emission of light (luminescence) is generally more sensitive than detection of absorption (as is the case with gold nanoparticles), the latter being performed against the higher background of transmitted light (for example, regarding organic fluorophors [9], [10] and [11]; regarding QDs: [11]-[17], [50]).

Unfortunately, these photoluminescent probes have several disadvantages, so that their potential as probes in strip tests cannot be fully exploited. Among these drawbacks, we may mention for example the phenomenon of photobleaching in the case of organic fluorophors which, following irreversible structural changes induced by illumination, is reflected in disappearance of the fluorescence, or else the phenomenon of twinkling of the emission for semiconductor nanocrystals, or “quantum dots”, the probes then periodically ceasing to emit, and consequently they are unsuitable for producing a constant, reproducible signal. Other drawbacks result for example from the width of the emission spectrum of the luminescent probes. In fact, an emission spectrum that is too broad makes it difficult to filter any background signal that may be present, and this affects the quality of the signal and, in particular, the signal to noise ratio. It is also necessary to take into account, in addition to the optical factors that contribute to the efficiency of the probe in a biological assay, the practical character and the ease of use of the probe. Thus, certain particles, as is the case with semiconductor nanocrystals, lose their luminescence characteristics after freezing, which represents a drawback for storage of bioconjugated semiconductor nanocrystals. The ease of coupling of the probes to the molecular compound allowing the desired molecules to be targeted is also an aspect to take into consideration when choosing a suitable probe. Thus, a certain number of particles, including semiconductor nanocrystals, are synthesized in organic solvents. It follows that use for biological applications requires additional steps of surface preparation for dispersing these particles in water, a process that may be complex to implement and unstable over time ([18]). In fact, the surface functionalizations used for semiconductor nanocrystals do not involve covalent bonding with the surface of the nanocrystals. The functionalization molecules may thus become detached and cause dissociation of the reagent specifically binding the analyte (antibody or some other) of the nanocrystal that serves as a probe. Thus, conjugation of these nanocrystals to the specific binding reagents of the analyte may take some weeks to some months, depending on the type of functionalization. However, commercial use of a capillary action test requires a stability of the order of two years after deposition of the probe-binding reagent conjugates on the test strip.

Another drawback of these luminescent probes is that excitation, necessary for detecting the luminescence, may cause the emission of parasitic light, which has the consequence of increasing the background signal and consequently reducing the signal to noise ratio. Various approaches have been proposed for eliminating, or at least reducing, this signal from parasitic emission, such as, for example, using luminescent nanoparticles containing chelates or complexes of lanthanide ions, combined with delayed detection of the luminescence; up-conversion nanoparticles; or else nanoparticles with persistent luminescence.

Thus, luminescent particles, loaded with chelates or complexes of lanthanides, have already been proposed as luminescent probes in capillary action tests.

For example, Zhang et al. ([19]) propose the use of silica nanoparticles loaded with lanthanide (Eu) chelates, as probes for detecting the bacterium Pantoea stewartii subsp. stewartii (Pss) in a migration strip test. It is stated that these probes make it possible to attain a limit of detection 100 times lower than that obtainable with conventional assays using gold particles. Moreover, Xia et al. ([20]) use silica particles loaded with europium chelate in a lateral flow assay for detecting a hepatitis B surface antigen (HBsAg).

We may also cite the works of Liang et al. ([21]) and Juntunen et al. ([22]), who propose using polystyrene microparticles loaded with europium chelate, in a lateral flow assay, for detecting alpha-fetoprotein (AFP) in a serum sample, or else for detecting prostate-specific antigen (PSA) and biotinylated bovine serum albumin (biotin-BSA). The documents WO 2013/013214 and WO 2014/146215 also propose the use of polystyrene nanospheres loaded with chelates of terbium and/or europium as probes in a lateral flow assay strip.

It has also been proposed, in document WO 2014/146215, to exploit the long-lifetime emission (of the order of 100 μs) of these nanoparticles containing chelates or complexes of lanthanide ions, for implementing delayed detection that makes it possible to avoid the parasitic emissions, the lifetime of which is generally of the order of 1-10 ns.

However, the luminescent particles loaded with chelates or complexes of lanthanides, used as luminescent probes, typically only contain a single lanthanide ion per chelate or complex. Each chelate or complex takes up space that is not negligible within the nano- or microparticle, which limits the number of emitting ions correspondingly for a given particle size. For example, a nanoparticle of 45 nm only contains of the order of 1000 chelates and emitting ions [47]. Moreover, synthesis of particles of this type containing complexes or chelates of lanthanide ions comprises at least two steps: synthesis of the complex or chelate, and then synthesis of the particle containing the chelates. Thus, synthesis of particles of this type is complex and therefore relatively expensive. Finally, the stability of particles of this type has also been questioned ([23]).

In recent years, another type of luminescent nanoparticles based on rare earths has been proposed as a luminescent probe in applications in bioimaging, and in particular in capillary action tests: these are nanoparticles of up-conversion phosphors, which emit visible light under excitation by infrared or near-infrared sources (for example, [24]-[29]). In the context of using these up-conversion nanoparticles, two photons are absorbed by the nanoparticle before luminescence emission is observed, which corresponds to the detected signal. As an example, we may mention the work of Niedbala et al. ([30]), which proposes lateral flow assay strips using probes of the UPT type (“Up-converting Phosphor Technology”), allowing a higher sensitivity of detection to be reached than in the enzyme-linked immunoabsorption assays. Up-conversion luminescent probes of this kind are used for example in the device for lateral flow assay proposed in the document US 2014/0170674.

These up-conversion phosphors have in particular the advantage, compared to the aforementioned luminescent probes, that they display resistance to the phenomenon of photobleaching and they have a low level of parasitic fluorescence causing background noise. In fact, as excitation takes place at a lower wavelength than the detection wavelength, emission due to the ancillary substances contained in the sample or to the porous solid support is practically nonexistent.

Unfortunately, these up-conversion phosphors have the major drawback that they have low quantum yields, and their efficiency decreases considerably for low optical power densities of the source of excitation, the luminescence being proportional to the square of the power density of the excitation. Moreover, a fairly wide zone comprising the test band, and optionally the control band, must be excited, which decreases the power density of the excitation for a given power. Consequently, reading the tests using luminescent probes of this kind requires complex equipment, combining laser diodes for excitation, with other elements such as lenses, filters, photomultipliers, preamplifiers, etc.

Finally, inorganic nanoparticles emitting persistent luminescence have also been proposed for avoiding the parasitic luminescence induced by the excitation. These inorganic nanoparticles are formed from a crystalline matrix containing lanthanide ions as dopants. The particular feature of the nanoparticles with persistent luminescence is that the dopants introduce trap states in the electronic structure of the crystal, and the excited charges are trapped there. Thus, luminescence emission by these nanoparticles can only take place after release of the charges from these trap states, said release taking place by thermal activation ([31]). Depending on the energy of these trap states in the electronic structure, i.e. depending on the depth of the trap, the thermal activation, and thus the lifetime of the emission, may reach hours or even days. It is thus possible to excite the nanoparticles, then insert the capillary action test device in a suitable reader after the excitation stops, and read the assay by detecting the luminescence in the absence of excitation, and therefore in the absence of parasitic emissions. For example, Paterson et al. ([32]) obtained a sensitivity of detection that was significantly improved relative to that obtained with gold nanoparticles (limit of detection about 10 times lower than that obtained with gold nanoparticles). This also makes it unnecessary to use an emission filter for reading.

However, this system has the drawback that it requires a long acquisition time of the signal from emission. In fact, as the emission by these nanoparticles takes place for several minutes, in particular for several hours, or even days, depending on the circumstances, it is necessary to wait the equivalent of this lifetime for collecting a non-negligible fraction of the number of photons emitted. Thus, in unit time, for example per second, the number of photons emitted will be low, which consequently requires lengthening the acquisition time of the luminescence to reach a high level of sensitivity. Such an acquisition time is contrary to the objective of the capillary action tests, which is to supply a rapid diagnosis. To counteract this problem, it is possible to increase the excitation power; however, this involves complex equipment, which will not satisfy the requirement for an assay system that is compact and inexpensive.

Finally, YVO₄ nanoparticles codoped with europium and with bismuth were employed in another variant of capillary action test ([33]). The presence of bismuth makes it possible to produce a shift of the absorption of the YVO₄ matrix which has an absorption peak at 280 nm due to the charge transfer transition O²—V⁵⁺ inside the vanadate ions VO₄³⁻, toward the visible, owing to the appearance of the charge transfer transition Bi³⁺—V⁵⁺, thus allowing more usual excitation sources, around 350 nm, to be used. The excitation is then transferred to the Eu³⁺ ions. However, this approach has the drawback of complex synthesis of these nanoparticles. In particular, it is difficult to produce a homogeneous solid solution of YVO₄ and BiVO₄, whose crystalline structures are different ([34]). This requires having recourse either to hydrothermal syntheses at high temperature, requiring more complex equipment (autoclave) ([33]), or to ripening processes ([34]).

Thus, none of the probes proposed to date for capillary action tests is completely satisfactory. In particular, there is still a need for a probe, compatible with use in a capillary action test device, making it possible to combine the advantages of a probe that is very luminous, of low complexity and inexpensive to synthesize, which provides far more sensitive detection than the capillary action systems based on gold nanoparticles, with a system for reading with the naked eye or with a reader that is compact and inexpensive.

The present invention has precisely the aim of proposing new luminescent probes that can be used in a capillary action test, and which meet this need.

More particularly, it proposes the use, as probes in a method of analysis by a capillary action test, of luminescent nanoparticles doped with rare earth ions, with controlled optical and physicochemical properties.

Thus, the invention describes an in vitro method for detecting and/or quantifying a biological or chemical substance of interest in a liquid sample, by a capillary action test, using, as probes, photoluminescent inorganic nanoparticles, of the following formula (I):

(A₁₋ₓLnₓ)ₐ(MₚO_{q}) (I)

in which:
M represents one or more elements capable of combining with oxygen (O), to form a crystalline compound;
Ln corresponds to one or more luminescent lanthanide ions;
A corresponds to one or more ions forming part of the crystalline matrix whose electronic levels are not involved in the luminescence process;
0<x<1; and
the values of p, q and a are such that the electrical neutrality of (A₁₋ₓLnₓ)ₐ(MₚO_{q}) is respected;

said method employing detection of the luminescence, with an emission lifetime shorter than 100 ms, of the nanoparticles, after one-photon absorption.

More particularly, according to a first of its aspects, the invention relates to an in vitro method for detecting and/or quantifying a biological or chemical substance of interest in a liquid sample, by a capillary action test, using, as probes, photoluminescent inorganic nanoparticles of the following formula (II):

A₁₋ₓLnₓVO_{4(1-y)}(PO₄)_{y} (II)

in which:
A is selected from yttrium (Y), gadolinium (Gd), lanthanum (La), lutetium (Lu), and mixtures thereof;
Ln is selected from europium (Eu), dysprosium (Dy), samarium (Sm), neodymium (Nd), erbium (Er), ytterbium (Yb), thulium (Tm), praseodymium (Pr), holmium (Ho) and mixtures thereof;
0<x<1, in particular 0.2≤x<0.6 and more particularly x has a value of 0.4; and
0.0≤y<1, in particular y has a value of 0;

said method employing detection of the luminescence, with an emission lifetime shorter than 100 ms, of the nanoparticles, after one-photon absorption, by excitation of the matrix at a wavelength less than or equal to 320 nm.

In particular, detection of luminescence is advantageously effected by excitation of the matrix AVO_{4(1-y)}(PO₄)_{y}, for example YVO₄, at a wavelength less than or equal to 300 nm, in particular between 250 and 300 nm.

According to the method of the invention, the signal detected thus corresponds to the luminescence emission by the photoluminescent nanoparticles after absorption of a single photon, in other words the emission at a wavelength greater than the excitation wavelength. The luminescence emission after absorption of a single photon differs in particular from the case of detection of the luminescence emission by particles for two-photon absorption, as is the case for the “up-conversion” particles mentioned above.

These nanoparticles are functionalized with recognition molecules (antibodies, nucleic acids, peptides, aptamers, etc.) that are capable of recognizing the substance to be analyzed, as is described hereunder.

In the sense of the invention, “analysis” of the substance in a sample covers the aspect of detection or qualitative characterization of the presence or absence of said substance, as well as the aspect of determination, or quantitative characterization of said substance.

The liquid sample may in particular be a biological sample, in particular any biological fluid or body fluid. It may be a sample taken from a human, for example selected from blood, serum, plasma, saliva, expectoration, nasal smear, urine, diluted fecal matter, vaginal smear or cerebrospinal fluid.

It may also be a solution containing biological molecules, chemical molecules or pathogenic viruses or bacteria, for example such as environmental samples or samples from agricultural and food products.

The method of the invention may be used in particular for detecting and/or quantifying molecules, proteins, nucleic acids, toxins, viruses, bacteria or parasites, in a sample, in particular in a biological sample.

It may for example be used for detecting the presence of biomarkers, antibodies, DNA and/or RNA, immunoglobulins (IgG, IgM, etc.), antigens, and the antigens may also be biomolecules making up a virus, a bacterium or a parasite, in a biological sample.

It may also be for example a molecule of interest for scientific police investigations, for example an illegal chemical substance such as a drug, or a substance of interest for defense (bioterrorism agents).

It may also be a substance of interest for food safety (pathogenic bacteria such as Salmonella, Listeria, or Escherichia coli, or viruses such as norovirus or allergens), or for the environment, for example a pollutant (pesticides).

The biological or chemical substance of interest that we aim to analyze by the capillary action test according to the invention is denoted more simply hereinafter by the expressions “substance to be analyzed” or “analyte”.

The use of the luminescent inorganic nanoparticles according to the invention as probes in a capillary action test device, for example such as in a test strip, proves particularly advantageous in several respects.

Firstly, the nanoparticles doped with rare earth ions, employed according to the invention, of formula (A₁₋ₓLnₓ)ₐ(MₚO_{q}) (I) described more precisely hereunder, for example nanoparticles of the type YVO₄:Eu or GdVO₄:Eu, YAG:Ce, in particular the nanoparticles of formula A₁₋ₓLnₓVO_{4(1-y)}(PO₄)_{y} (II), have particularly advantageous properties, in particular with respect to their excellent photostability, which allows the acquisition of a prolonged, constant signal, and absence of a phenomenon of twinkling of the emission.

Moreover, these nanoparticles do not lose their luminescence after freezing.

Nanoparticles based on yttrium vanadate doped with rare earths have for example been described in detail by Riwotzki et al. ([45]) and Huignard et al. ([46]). As for document EP 1 282 824, it describes the use of surface-modified inorganic luminescent nanoparticles, as probes for detecting a biological substance or some other organic substance.

However, as far as the inventors know, it has never been proposed to take advantage of photoluminescent inorganic nanoparticles as defined above, doped with rare earth ions, different than particles with persistent luminescence, and emitting after absorption of a single photon, for use as luminescent probes in a capillary action test.

It was not in any way foreseeable that these nanoparticles based on lanthanide ions could be used for detecting and quantifying chemical or biological substances, in particular in a capillary action test and, furthermore, that they would lead to improved performance in terms of sensitivity of the test.

In fact, apart from the absence of twinkling, the luminescence properties of the nanoparticles based on rare earths are considered to be inferior to those of quantum dots. In these nanoparticles doped with rare earth ions, in particular those consisting of a metal oxide matrix, excitation of the luminescence may be done either by direct excitation of the matrix, or, less often, by direct excitation in the visible, of the luminescent rare earth ions. The extinction coefficient for the direct absorption of the rare earth ions is in general very low, but the extinction coefficient for excitation of the crystalline matrix is much higher ([35]).

However, the absorption band of the crystalline matrix is generally located in the UV, which presents a major drawback: the biomolecules as well as the various components of the capillary action device (for example, the capillary diffusion membrane) also absorb strongly in the UV. For example, in the case of a matrix based on vanadate ions VO₄³⁻, the absorption peak at 280 nm coincides with the absorption of proteins, and of tryptophan amino acids in particular. Consequently, excitation of the luminescence of nanoparticles based on a crystalline matrix doped with rare earth ions, in particular in the UV, is liable to produce large subsidiary emission signals. Parasitic emission like this is incompatible with the objective of an in vitro diagnostic test such as a capillary flow strip test, which aims precisely to identify a signal of low intensity from a complex mixture of molecules.

Against all expectations, the inventors discovered that it is possible to use these photoluminescent nanoparticles based on rare earths according to the invention, as probes in an in vitro diagnostic technique of the capillary action test type, even in conditions of excitation in the UV and in particular for excitation below 350 nm, advantageously below 320 nm, more advantageously between 250 and 320 nm and in particular between 250 and 300 nm.

Without wishing to be bound to a theory, the inventors found that detection, in a capillary action test, of the luminescence of the nanoparticles excited in the UV proves possible, despite the presence of a strong signal from parasitic emissions, owing to three optical properties, specific to the nanoparticles employed: i) a large number of luminescent lanthanide ions contained in the nanoparticles of the invention without the necessity of having recourse to nanoparticles of very large size, ii) a narrow emission spectrum of the rare earth ions, which makes it possible to eliminate the parasitic emissions effectively, which in general are very wide spectrally and iii) a large Stokes shift (shift between the absorption peak and the emission peak), typically of the order of 350 nm for YVO₄ or GdVO₄ nanoparticles doped with Eu (absorption peak at 280 nm for the vanadate matrix and emission peak of Eu at 617 nm), which allows effective rejection of the excitation wavelengths and of the parasitic emissions due to the migration support or to the sample containing the substance to be analyzed, which generally have a small Stokes shift. In particular, this large Stokes shift means that for detection it is possible to use a simple high-pass filter, which is less expensive than an interference filter.

Therefore it is possible to obtain effective elimination of the parasitic emissions, and acquisition of a signal with a signal to noise ratio sufficient to attain the desired sensitivity of detection.

Moreover, it was found, against all expectations, that excitation below 320 nm and in particular around 280 nm, where the absorption peak of the vanadate matrix is located (see FIG. 11(A)) or at 300 nm, induces parasitic emissions, linked to the nitrocellulose membrane typically used for lateral flow assays, very much lower than excitation at 380 nm (see FIG. 11(B)). Knowing that the molecules chelating or complexing the lanthanide ions typically absorb between 320 nm ([47]) and 400 nm ([48]), the use of a matrix absorbing between 250 and 300 nm, in particular between 260 and 300 nm, offers an additional advantage.

In particular, besides the O²⁻—V⁵⁺ charge transfer absorption within the vanadate ions VO₄³⁻ in a crystal of YVO₄ or GdVO₄, which is centered at 280 nm, or in a crystal of LaVO₄, centered around 300 nm ([40]), in the case of nanoparticles containing Eu, another absorption, linked to an O²⁻-Eu³⁺ charge transfer, is possible and leads to absorption centered around 260 nm. The latter has for example been observed in La₂Hf₂O₇:Eu, A₂Hf₂O₇ (A=Y, Gd, Lu) and La₂Zr₂O₇:Eu nanoparticles ([41]).

Furthermore, as illustrated in the examples given hereunder, the method of the invention makes it possible to reach performance of the capillary action test, in terms of sensitivity of detection, that is improved by at least one order of magnitude, or even much more.

In particular, it not only makes qualitative analyses possible (presence or absence of the analyte in the sample), but also semiquantitative and quantitative analyses.

Thus, the invention further relates to the use, as probes in a capillary action test device, of nanoparticles as defined above, to increase the sensitivity of detection of said capillary action test device.

The photoluminescent nanoparticles may be employed, as probes, in any known type of capillary action test, for example lateral flow assays, whether it is a so-called “sandwich” assay as shown schematically in FIG. 2, or a so-called “competitive” assay as shown in FIG. 3. In particular, they are suitable for use in capillary action test devices proposed to date with gold nanoparticles as luminescent probes, without having to modify the characteristics of the support of the capillary action test device.

In particular, as illustrated in the examples given hereunder, the photoluminescent nanoparticles according to the invention may for example have an average size similar to that of the gold nanoparticles, of the order of 30 to 50 nm, and consequently compatible with the capillary action means, typically a nitrocellulose membrane, suitable for migration of particles of this size.

Alternatively, the photoluminescent nanoparticles according to the invention may be larger, thus making it possible to optimize the luminescence signal. In fact, the number of lanthanide ions increases with the volume of the nanoparticle and thus the luminescence signal emitted increases with the cube of the radius of a spherical particle. In this case, the migration support of the lateral flow assay may contain pores adapted to the size of the nanoparticles selected. Membranes with variable pore sizes are for example commercially available (for example, the membranes with variable pore sizes marketed under the references HF075, HF090, HF120, HF135, HF180, by the company MerckMillipore).

Larger nanoparticles may be obtained for example by sorting for size by centrifugation of the particles as exemplified, only keeping, in the size distribution, the particles of the largest sizes, or may be obtained by grinding the bulk material. Any other technique known by a person skilled in the art may also be used.

Moreover, as illustrated in the examples, while maintaining a particle size similar to that of the gold particles, the nanoparticles of the invention have a large number of ions giving rise to luminescence, in particular significantly greater than in the case of particles based on chelates or complexes of lanthanides, and thus make it possible to produce an emission signal of high intensity, and consequently achieve improved sensitivity.

Advantageously, the method of detection according to the invention makes qualitative measurement possible up to 10 times, in particular up to 100 times, or even up to 1000 times, lower than the limit of detection of one and the same assay employing gold nanoparticles as probes.

Finally, the use of the nanoparticles according to the invention as probes in a capillary action test even leads to improved performance, in terms of sensitivity, compared to the results obtained with particles loaded with chelates of lanthanides.

According to another of its aspects, the invention relates to a capillary action test device, useful for detecting and/or quantifying a biological or chemical substance of interest in a liquid sample, said device comprising, as probes, photoluminescent inorganic nanoparticles as defined above.

More precisely, the invention thus relates to a capillary action test device, useful for detecting and/or quantifying a biological or chemical substance of interest in a liquid sample, said device comprising, as probes, photoluminescent inorganic nanoparticles of formula A₁₋ₓLnₓVO_{4(1-y)}(PO₄)_{y} (II), in which A, Ln, x and y are as defined above, said nanoparticles being able to emit luminescence, with an emission lifetime shorter than 100 ms, after one-photon absorption, by excitation of the matrix at a wavelength less than or equal to 320 nm, in particular less than or equal to 300 nm and more particularly between 250 and 300 nm.

Thus, the capillary action test device according to the invention comprises photoluminescent inorganic nanoparticles of formula (II), the luminescence of which is detected, with an emission lifetime shorter than 100 ms, after one-photon absorption, by excitation of the matrix at a wavelength less than or equal to 320 nm, in particular less than or equal to 300 nm and more particularly between 250 and 300 nm.

More precisely, like the probes, for example gold nanoparticles, used conventionally in the known devices for lateral flow assay, the photoluminescent nanoparticles according to the invention are present, in particular at the level of a zone of the assay device called “labeling zone” (more commonly called “Conjugate Pad” in English-language terminology), in a form coupled to at least one reagent specifically binding the substance to be analyzed, such as an antibody.

The invention is described more particularly hereunder with reference to a conventional capillary action test device of the migration strip type (known by the English-language designation “Lateral Flow Strip”), as shown schematically in FIG. 1. Of course, the method of the invention may employ any other variant of capillary action test device, provided that it is suitable for using the photoluminescent nanoparticles of the invention, as probes.

Typically, a capillary action test device according to the invention may thus comprise a means for capillary action in a reference direction, in particular a porous solid support, such as a nitrocellulose membrane, comprising:
a zone for deposition of the liquid sample;
a zone, arranged downstream of the zone for deposition of the sample, called labeling zone or coupling zone, loaded with the photoluminescent inorganic nanoparticles according to the invention (probes), coupled to at least one “binding reagent” (recognition molecule), for example an antibody, specific to the substance to be analyzed;
a reaction zone, also called “detection zone”, arranged downstream of the labeling zone, in which at least one “capturing reagent” (recognition molecule) is immobilized, such as an antibody, specific to the substance to be analyzed;
a control zone of migration of the reagents, located downstream of the detection zone; and

optionally, an absorbent pad, arranged downstream of the control zone.

Examples of devices for capillary action tests will be described in more detail hereunder.

The liquid sample may be analyzed directly using the capillary action test device according to the invention. The analysis of a liquid sample according to the method of the invention typically comprises:

(i) applying the liquid sample to be analyzed, and optionally a diluent, at the level of the deposition zone of the capillary action test device;

(ii) incubating the device until the luminescence generated by the photoluminescent nanoparticles is detected in the reaction zone and/or until the luminescence is detected in the migration control zone; and

(iii) reading and interpreting the results.

According to another of its aspects, the present invention relates to the use of a capillary action test device according to the invention for detecting and/or quantifying a biological or chemical substance of interest in a liquid sample, in particular a biological sample.

The capillary action test device may be coupled to a reader, which supplies the test result.

As detailed hereunder, reading of the results comprises detecting the luminescence generated by the nanoparticles immobilized at the level of the detection zone, and if applicable at the level of the control zone, of the capillary action test device.

It is carried out more particularly by:
excitation of the immobilized photoluminescent nanoparticles; and
detection of the luminescence emission.

Particularly advantageously, it is possible to read the capillary action test device with the naked eye, using only a suitable filter.

Alternatively, the luminescence may be read using simple detection equipment, for example using an emission filter and a detector such as a camera.

The emission filter may be an interference filter but may also be a simple high-pass filter. In fact, owing to the large Stokes shift associated with the emission of these particles, any parasitic emission, which generally has a small Stokes shift, will be located at shorter wavelength than the emission from these nanoparticles.

Finally, the capillary action test device according to the invention is suitable for multiplexed detection, in other words for the simultaneous detection, by the same capillary action test, of several substances in one and the same sample.

According to another of its aspects, the invention further relates to the use of a method of detection as defined above, or of a capillary action test device as defined above, for purposes of in vitro diagnostics. Advantageously, the possibility, with the capillary action test according to the invention, of detecting low contents of certain substances in biological samples makes it possible, for example, to use the method of the invention for earlier detection of diseases, or diagnosis of the evolution of a disease or the effect of a therapeutic treatment.

The diseases that can be diagnosed by a capillary action test according to the invention are not limited and comprise all diseases revealed by the presence of a specific marker of the disease, of the molecule of biological interest type (protein, nucleic acid, antibody, etc.), for which there are one or more specific binding partners (ligand, antibodies, antigens, complementary nucleic acids, aptamers, etc.).

As examples, we may mention infectious diseases (bacterial, parasitic, or viral, such as AIDS), inflammatory and autoimmune diseases, cardiologic, neurological, or oncologic diseases (for example, solid cancers such as breast cancer or prostate cancer).

The largest gains in sensitivity (by a factor of 100 or 1000) make it possible to achieve performance close to an ELISA assay or one of the variants of ELISA. Thus, the method of the invention is particularly suitable in cases when sensitive detection of the ELISA type is necessary, but unavailable.

It thus makes rapid diagnosis possible, at the point of care (POC).

The method of the invention may thus be useful for the diagnosis of infectious diseases or other common diseases, for example in developing countries, in rural and/or remote areas for the diagnosis of infectious diseases or other common diseases.

It may also prove particularly useful in the context of emergency care (SAMU, SMUR emergency medical services), to allow urgent diagnosis, in particular in the case when the patient's survival may be in jeopardy (heart failure, venous thrombosis, inflammatory syndrome, systemic bacterial infection (sepsis), acute pancreatitis). In such situations, it may be used for carrying out a rapid assay with sensitivity comparable to that of an ELISA assay prior to arrival at the hospital, saving time in diagnosis and management of the patient, and thus improving the patient's chances of survival.

Moreover, diagnosis by a capillary action test, using particles according to the invention as probes, may be particularly useful for patients who require regular diagnostic tests for adjusting the dose of medicinal products administered (for example in the case of immunomodulators or immunosuppressants). In fact, carrying out a strip test, rather than diagnosis by taking a blood sample or other more invasive examination, advantageously makes it possible to improve comfort for the patient, reduce the costs of diagnosis, perform detection/quantification closer in time, and thus allow better adjustment of the doses of medicinal products administered.

Of course, the method of the invention is not limited to the applications mentioned above. Thus, it may be used for detecting nucleic acids (GMOs in seeds for example), or for detecting a pollutant or a pathogen in the environment, for example in water, or in foodstuffs intended for human or animal consumption.

The applications of the method of the invention may thus extend from immunology to molecular genetics or to detection of DNA and RNA. It may be used for labeling one or more strands of RNA of a biological sample, with a partially complementary fragment bound to a nanoparticle, and then detect them by hybridization on complementary fragments of another region grafted on the substrate of a strip, following an approach similar to the DNA chips of the Affymetrix type. One advantage of the invention is the absence of an amplification step that is usually necessary for these approaches.

The method of the invention may also be used for detecting illegal chemical substances, for example drugs or any other substance of interest for the police or defense.

It may also be used for detecting and/or quantifying a substance of interest, in particular a pathogen, in an agricultural or food product or in the environment.

Other features, variants and advantages of the method and of the capillary action test device according to the invention will become clearer on reading the description, examples and figures, given hereunder for purposes of illustration, and not limiting the invention.

Hereinafter, the expressions “between . . . and . . . ”, “ranging from . . . to . . . ” and “varying from . . . to . . . ” are equivalent and are intended to signify that the limits of the range are included, unless stated otherwise.

Unless stated otherwise, the expression “comprising a/one” is to be understood as “comprising at least one”.

### Photoluminescent Inorganic Nanoparticles

As stated above, the method of the invention employs, as probes in a capillary action test device, photoluminescent inorganic nanoparticles having specific optical and physicochemical properties.

The photoluminescent nanoparticles of the invention are formed from a crystalline matrix doped with rare earth ions. The “crystalline matrix” is typical of a crystalline solid, in which certain atoms are replaced by other atoms, called “substituted ions”. The substituted ions make it possible to modify a chemical or physical property of the crystalline matrix, in particular to endow the nanoparticle with a quality of optical emission.

The rare earth ions in the nanoparticles of the invention are not in the form of complexes or chelates of rare earth ions, the latter being formed from rare earth ions in combination with suitable organic ligands, for example as described in the work by Yuan et al. ([36]).

The nanoparticles of the invention may be doped with rare earth ions of the same nature or of different natures.

More particularly, they may be lanthanide ions selected from europium (Eu), dysprosium (Dy), samarium (Sm), praseodymium (Pr), neodymium (Nd), erbium (Er), ytterbium (Yb), cerium (Ce), holmium (Ho), terbium (Tb), thulium (Tm) and mixtures thereof.

In particular, the lanthanide ions may be selected from Eu, Dy, Sm, Pr, Nd, Er, Yb, Ho, Tm and mixtures thereof, in particular from Eu, Dy, Sm, Yb, Er, Nd and mixtures thereof, in particular from Eu, Dy, Sm and mixtures thereof, and in particular Eu.

The luminescent inorganic nanoparticles used as probes in a capillary action test according to the invention are more particularly of the following formula (I):

(A₁₋ₓLnₓ)ₐ(MₚO_{q}) (I)

in which:
M represents one or more elements capable of combining with oxygen (O), to form a crystalline compound;
Ln corresponds to one or more luminescent lanthanide ions;
A corresponds to one or more ions forming part of the crystalline matrix whose electronic levels are not involved in the luminescence process;
0<x<1, in particular 0.1≤x≤0.9, in particular 0.2≤x≤0.6, in particular 0.2≤x≤0.4 and more particularly x has a value of 0.4; and
the values of p, q and a are such that the electrical neutrality of (A₁₋ₓLnₓ)ₐ(MₚO_{q}) is respected;

said method employing detection of the luminescence, with an emission lifetime shorter than 100 ms, of the nanoparticles, after one-photon absorption, in other words detection of the luminescence at a wavelength greater than the excitation wavelength.

A may be selected more particularly from yttrium (Y), gadolinium (Gd), lanthanum (La), lutetium (Lu) and mixtures thereof; in particular A represents Y, Gd or La, in particular Y or Gd; and preferably A represents Y.

In particular, M in the aforementioned formula (I) may represent one or more elements selected from V, P, W, Mo, As, Al, Hf, Zr, Ge, Ti, Sn and Mn. The crystalline matrix of the nanoparticles used according to the invention may incorporate one or more types of anions MₚO_{q}.

Preferably, M represents one or more elements selected from V, P, Al, Hf, Zr, Ge, Ti, Sn and Mn.

In particular, M may represent V_{1-y}P_{y}, with y ranging from 0 to 1. More particularly, M may represent V.

According to a particular embodiment, p in the aforementioned formula (I) is different from zero.

As an example, a nanoparticle of the invention may be of formula (I) in which M represents V and/or P, p has a value of 1, so that the matrix Aₐ(MₚO_{q}) of said nanoparticle comprises VO₄³⁻ and/or PO₄³⁻ anions.

According to another particular embodiment, a nanoparticle of the invention may be of formula (I) in which M represents Hf or Zr, Ge, Ti, Sn, Mn, p has a value of 2 and q has a value of 7, so that the matrix of said particle is AₐHf₂O₇, AₐZr₂O₇, AₐGe₂O₇, AₐTi₂O₇, AₐSn₂O₇ or AₐMn₂O₇. In particular, A may represent La, Y, Gd or Lu, in which case a=2.

In another embodiment example, M represents A1, A represents Y or Lu, p has a value of 5 and q has a value of 12, so that the matrix Aₐ(MₚO_{q}) of said nanoparticle is garnet Y₃Al₅O₁₂ (YAG) or Lu₃Al₅O₁₂ (LuAG).

In another embodiment example, p has a value of zero and A represents Y or Gd, so that the matrix Aₐ(MₚO_{q}) of said nanoparticle is of the type Y₂O₃ or Gd₂O₃.

Thus, according to a variant embodiment, the luminescent inorganic nanoparticles used in a capillary action test are of formula Gd₂O₃:Ln, in which Ln represents one or more luminescent lanthanide ions, in particular as defined above, the level of doping of the nanoparticles with Ln ions being between 10 and 90%, in particular between 20 and 60%, in particular between 20 and 40% and more particularly 40%.

In the context of this variant embodiment, the luminescence may be detected by excitation of the matrix at a wavelength below 250 nm [51].

The degree of substitution of the ions of the crystalline matrix of the nanoparticles according to the invention, in particular of the metal oxide matrix, with rare earth ions may more particularly be between 10% and 90%, in particular between 20 and 60%, in particular between 20 and 40% and more particularly 40%.

It was counterintuitive to select such high levels of doping. In fact, in general, the usual levels of doping of the luminescent nanoparticles doped with rare earth ions are maintained at values below 10% to avoid the “quenching” effect that occurs at higher concentrations ([42]-[44]).

Advantageously, the imperfect crystallinity of the nanoparticles according to the invention, as described in more detail hereunder, allows the “quenching” effect to be avoided.

According to another of the characteristics of the photoluminescent nanoparticles according to the invention, they are able to emit luminescence after absorption of a single photon, which corresponds to the detected signal.

Moreover, the luminescence emission by the nanoparticles of the invention, in contrast to the so-called nanoparticles with persistent luminescence ([32]), does not involve “trap” states.

Thus, the nanoparticles of the invention have a luminescence emission lifetime shorter than 100 ms, in other words strictly less than 100 ms ([35], [39], [49]).

The emission lifetime is to be understood as the lifetime of the excited state of the emitting nanoparticle, and more specifically of the emitting rare earth ions, and is determined in practice by the duration of the luminescence emission photons after cessation of the excitation, or the characteristic time of the exponential decay of luminescence after cessation of the excitation.

The emission lifetime of an emitting nanoparticle is different than the luminescence emission time before photodegradation or photobleaching of the nanoparticles.

The nanoparticles used according to the invention more particularly have an emission lifetime less than 100 ms, or even less than 10 ms, or even less than 1 ms.

Advantageously, the nanoparticles used according to the invention have an emission lifetime greater than or equal to 5 μs, in particular greater than or equal to 10 μs, in particular greater than or equal to 20 μs, or even greater than or equal to 50 μs.

It is possible to take advantage of the emission lifetime of the particles of the invention (some hundreds of μs in the case of the Y₁₋ₓEuₓVO₄ particles, compared to the lifetimes of the usual fluorophors of the order of a nanosecond), to carry out time-resolved detection, in particular delayed detection of the emission, with a sufficient temporal resolution (of the order of 10 μs or even of the order of 100 μs), using unsophisticated and inexpensive material. For example, it is possible to modulate the current of an LED (Light Emitting Diode) that may be used for excitation and record a series of images of the strip instead of just one and then analyze the signal as a function of time so as to eliminate any residual parasitic emission of short lifetime (of the order of 10 ns or less).

According to a variant embodiment, the photoluminescent nanoparticles used according to the invention may be of the following formula (II):

A₁₋ₓLnₓVO_{4(1-y)}(PO₄)_{y} (II)

in which:
A is selected from yttrium (Y), gadolinium (Gd), lanthanum (La), lutetium (Lu) and mixtures thereof, in particular A represents Y;
Ln is selected from europium (Eu), dysprosium (Dy), samarium (Sm), neodymium (Nd), erbium (Er), ytterbium (Yb), thulium (Tm), praseodymium (Pr), holmium (Ho) and mixtures thereof, preferably Ln represents Eu;
0<x<1, in particular 0.2≤x≤0.6 and more particularly x has a value of 0.4; and
0≤y<1, in particular y has a value of 0 said method employing detection of the luminescence, with a lifetime shorter than 100 ms, from the nanoparticles, after one-photon absorption.

According to a particular embodiment, the nanoparticles used according to the invention correspond to the aforementioned formula (II) in which y has a value of 0. In other words, the nanoparticles may be of formula A₁₋ₓLnₓVO₄ (III), in which A, Ln and x are as defined above.

A, in the aforementioned formula (II) or (III), may more particularly be selected from yttrium (Y), gadolinium (Gd), lanthanum (La) and mixtures thereof. In particular, A represents Y or Gd. According to a particular embodiment, A in the aforementioned formula (II) or (III) represents yttrium (Y).

Ln, in the aforementioned formula (II) or (III) may more particularly be selected from europium (Eu), dysprosium (Dy), samarium (Sm), ytterbium (Yb), erbium (Er), neodymium (Nd) and mixtures thereof. In particular, Ln is selected from Eu, Dy, Sm and mixtures thereof. According to another particular embodiment, Ln in the aforementioned formula (II) or (III) represents Eu.

Thus, according to a variant embodiment, the nanoparticles used as luminescent probes according to the invention are of formula Y₁₋ₓEuₓVO₄ (IV) in which 0<x<1, in particular 0.2≤x≤0.6 and more particularly x has a value of 0.4.

According to a variant embodiment, it is possible to exploit the direct absorption of the rare earth ions, in cases when the corresponding electronic transition is permitted. In these cases, the direct absorption is stronger than when the corresponding electronic transition is forbidden even if it generally remains weaker than the absorption of the oxide matrix. Two examples of rare earth ions to which this case applies are the Eu²⁺ and Ce³⁺ ions. These ions may for example be present as constituents of the following inorganic nanoparticles: LaPO₄ or YAG in the case of Ce³⁺ and Sr₂O₄ in the case of Eu²⁺.

The photoluminescent nanoparticles used according to the invention may have an average size greater than or equal to 5 nm and strictly less than 1 μm, in particular between 10 nm and 500 nm, preferably between 20 nm and 200 nm and in particular between 20 nm and 100 nm.

The photoluminescent nanoparticles used according to the invention thus have a sufficient volume to contain a large number of rare earth ions, and therefore emit a sufficient luminescent signal to allow detection of low concentrations of analyte.

Preferably, the nanoparticles of the invention comprise at least 10³ rare earth ions, in particular between 1000 and 6 000 000 rare earth ions, in particular between 5000 and 500 000 and more particularly between 20 000 and 100 0000 rare earth ions.

As an example, a Y_{0.6}Eu_{0.4}VO₄ spherical nanoparticle with a diameter of 30 nm contains 70 000 Eu³⁺ ions (calculation of the number of ions according to the reference Casanova et al. [37]).

The average size can be measured by transmission electron microscopy. The images from transmission electron microscopy make it possible to determine the shape of the nanoparticles (spherical, ellipsoidal) and deduce the average dimensions of the nanoparticles. In the case of particles that are spherical overall, the average size means the average diameter of the particles. In the case of particles of ellipsoidal shape, the average size means the average size of a sphere with the same volume as the ellipsoid. It is generally assumed that the third axis of the ellipsoid, not visible in the transmission images, which are 2D projections, is equal in length to the axis of the smallest size.

According to a particular embodiment, the nanoparticles of the invention are of elongated (prolate) overall ellipsoidal shape.

They may more particularly have a length of the major axis, designated a, between 20 and 60 nm; and a length of the minor axis, designated b, between 10 and 30 nm. In particular, the nanoparticles of the invention may have an average value of length of the major axis, a, of 40 nm and an average value of length of the minor axis, b, of 20 nm.

Advantageously, the nanoparticles used according to the invention have a low polydispersity. It is preferable for the polydispersity index, which may be deduced from the measurements of dynamic light scattering, to be strictly below 0.2. When this is not the case at the end of synthesis or functionalization of the particles, a lower polydispersity may be obtained by sorting for size by centrifugation or by any other technique known by a person skilled in the art.

According to a particular embodiment, the product of the level of doping with rare earth ions, for example with europium (Eu), times the quantum efficiency of the emission by the nanoparticle is maximized.

The product of the level of doping x with Ln ions times the quantum efficiency can be maximized using strong doping with Ln ions, for example between 0.2 and 0.6, and in particular 0.4, but without decreasing the quantum efficiency, in particular by limiting the transfer processes between doping ions, leading to an extinction of concentration. In particular, in order to maintain a high quantum efficiency, the nanoparticle has imperfect crystallinity. In fact, excellent crystallinity promotes the transfer processes between doping ions, especially when the latter are close together, as is the case for high levels of doping, and consequently promotes the processes of deexcitation of the ions by nonradiative processes, linked to the surface and the presence of the solvent. In particular, a method of synthesis at room temperature, or at least at a temperature not exceeding 600° C., is favorable for the imperfect crystallinity required for these nanoparticles.

The crystallinity of the nanoparticles is considered to be “imperfect” when the coherence length, determined by the X-ray diffraction pattern in at least one given crystallographic direction, is less than 80% of the particle size in that direction as measured from the transmission electron microscopy images. Different types of imperfect crystallinity may be considered: polycrystallinity, defects, porosity, etc.

Advantageously, the nanoparticles used according to the invention are each able to emit more than 10⁸ photons before emission ceases, in particular more than 10⁹, or even more than 10¹⁰ photons. In a great many cases, in particular in the case of YVO₄ or GdVO₄ particles doped with Eu, no cessation (extinction) of emission is observed under continuous illumination. In other words, advantageously, the nanoparticles according to the invention do not display phenomena of irreversible photodegradation or photobleaching.

Advantageously, the nanoparticles used according to the invention display good colloidal stability in solution.

The stability of the nanoparticles in solution is particularly decisive for meeting the requirements in terms of reproducibility of the results of detection based on use of these particles as probes in a capillary action test device.

In particular, good colloidal stability of the nanoparticles makes it possible to ensure, during migration of the liquid sample in the porous support of the capillary action test, migration of the luminescent nanoparticles, if applicable bound to the substance to be analyzed, as far as the detection zone and, optionally, as far as the control zone of the device.

The “zeta potential” is one of the elements representative of the stability of a suspension. It may for example be measured directly using equipment of the Zetasizer Nano ZS type from the Malvern company. Using optical devices, this equipment measures the speeds of displacement of the particles as a function of the electric field applied on them.

In particular, the nanoparticles of the invention advantageously have, at the end of synthesis, a zeta potential, designated ζ, less than or equal to −28 mV, in an aqueous medium at pH≥5. In particular, the nanoparticles have a zeta potential ζ, in an aqueous medium at pH≥6.5, in particular at pH≥7, and in particular at pH≥8, less than or equal to −30 mV.

The “zeta potential”, designated ζ, may be defined as the potential difference that exists between the bulk of the solution, and the shear plane of the particle. It is representative of the stability of a suspension. The shear plane (or hydrodynamic radius) corresponds to an imaginary sphere around the particle in which the solvent moves with the particle when the particles move in the solution. The zeta potential can be determined by methods known by a person skilled in the art, for example by displacement of the particle with its solubilization layer in an electric field.

This negative zeta potential of the nanoparticles, less than or equal to −28 mV at pH≥5, increases the phenomena of electrostatic repulsion of the nanoparticles in aqueous solution relative to one another, which thus allows the flocculation phenomena to be suppressed. It is in fact known empirically by a person skilled in the art that a zeta potential of high absolute value, in particular above 28 mV, generally allows the flocculation effects to be suppressed in media with low ionic strength.

It is to be understood that measurements of the zeta potential are carried out after purification of the aqueous suspension of the particles, and therefore for an aqueous suspension having an ionic conductivity strictly below 100 μS·cm⁻¹. The ionic conductivity of the suspension, allowing the level of ions present in said suspension to be estimated, can be measured, at room temperature (25° C.), with any known conductivity meter.

According to a particular embodiment, the luminescent nanoparticles employed according to the invention may have one or more surface molecules, promoting keeping them in suspension, owing to a high zeta potential.

According to a particular embodiment, the nanoparticles used according to the invention may have tetraalkylammonium cations on the surface. Nanoparticles of this kind, and their method of synthesis, are described for example in the application filed under No. FR1754416.

As an example, the photoluminescent nanoparticles used according to the invention may be of formula Y_{0.6}Eu_{0.4}VO₄, on the surface of which tetramethylammonium cations are optionally immobilized.

The nanoparticles used according to the invention, in particular the nanoparticles of the aforementioned formula (II), are predominantly of a crystalline and polycrystalline nature, in particular with an average size of crystallites, deduced by X-ray diffraction, between 3 and 40 nm.

Preparation of the Nanoparticles

The photoluminescent nanoparticles with a crystalline matrix doped with rare earth ions employed according to the invention can be prepared by any conventional method known by a person skilled in the art.

In particular, they may be prepared by a colloidal synthesis route. The methods of aqueous colloidal synthesis are familiar to a person skilled in the art (Bouzigues et al., ACS Nano 5, 8488-8505 (2011) [49]). These syntheses in an aqueous medium have the advantage of not requiring any subsequent step of solvent transfer.

As an example, the nanoparticles of formula A₁₋ₓLnₓVO_{4(1-y)}(PO₄)_{y} (II) may be formed by a coprecipitation reaction, in an aqueous medium, starting from precursors of said elements A and Ln, and starting from precursors of orthovanadate ions (VO₄³⁻) and optionally of phosphate ions (PO₄³⁻).

The precursors of the elements A and Ln may, conventionally, be in the form of salts of said elements, for example nitrates, chlorides, perchlorates or acetates, in particular nitrates. The precursors of the elements A and Ln, and the amount thereof, are of course selected in a suitable manner having regard to the desired nature of the nanoparticle.

For example, synthesis of nanoparticles of formula Y₁₋ₓEuₓVO₄ (IV) may employ, as precursor compounds of yttrium and europium, yttrium nitrate (Y(NO₃)₃) and europium nitrate (Eu(NO₃)₃).

A method of synthesis by the colloidal route of this kind, for photoluminescent nanoparticles used according to the invention, is described for example in the application filed under No. FR1754416. Advantageously, as described in application No. FR1754416, the coprecipitation reaction may be carried out in the presence of an effective amount of tetraalkylammonium cations.

The synthesis of luminescent nanoparticles according to the invention, in particular of larger sizes, greater than some tens of nanometers, may be accomplished by any other approach known by a person skilled in the art, for example by grinding.

Surface Functionalization of the Luminescent Nanoparticles

Coupling of the Nanoparticles to a Binding Reagent

Like the probes used conventionally in lateral flow assay devices, the luminescent nanoparticles employed according to the invention are coupled to at least one binding reagent specific to the substance to be analyzed.

The function, and consequently the nature, of the binding reagent coupled to the luminescent probes vary depending on the nature of the capillary action test, in particular lateral flow assay, employed, as detailed hereunder, in particular depending on whether it is a so-called “sandwich” assay or a “competitive” assay.

Thus, “binding reagent” means any chemical, biochemical or biological compound capable of binding specifically to the biological or chemical substance of interest that is required to be identified, in the context of an assay of the “sandwich” type, or to the capturing reagent of the detection zone competing with the biological or chemical substance of interest whose identification is required in the context of a “competitive” assay. As detailed hereunder, the binding reagent is also capable of binding specifically to the second capturing reagent immobilized in the control zone of the device for lateral flow assay.

“Bind” or “binding” means any strong bond, for example covalent, or, preferably, a collection of weak bonds, for example of the antigen/antibody type.

The nature of the binding reagent coupled to the luminescent nanoparticles employed as probes according to the invention is of course selected having regard to the substance to be analyzed in the sample.

Advantageously, the photoluminescent nanoparticles used according to the invention are perfectly suitable for a great variety of biological targeting, the specific results being dependent on the nature of the binding reagent or reagents grafted on the surface of the nanoparticle.

The binding reagent may more particularly be selected from a polyclonal or monoclonal antibody, an antibody fragment, a nanobody, an antigen, an oligonucleotide, a peptide, a hormone, a ligand, a cytokine, a peptidomimetic, a protein, a carbohydrate, a chemically modified protein, a chemically modified nucleic acid, a chemically modified carbohydrate that targets a known cell surface protein, an aptamer, an assembly of proteins and DNA/RNA or a chloroalkane used in labeling of the HaloTag type. An approach of the SNAP-Tag or CLIP-Tag type may also be used.

According to a particular embodiment, it is an antibody or antibody fragment, a peptide, a chemically modified nucleic acid or an aptamer, in particular an antibody.

Suitable antibody fragments comprise at least one variable domain of an immunoglobulin, such as simple variable domains Fv, scFv, Fab, (Fab′)² and other proteolytic fragments or “nanobodies” (antibodies with a single domain such as V_{H}H fragments obtained from antibodies of the camel family or V_{NAR} obtained from antibodies of cartilaginous fishes).

The term “antibodies” according to the invention includes chimeric antibodies, human or humanized antibodies, recombinant and modified antibodies, conjugated antibodies, and fragments thereof.

The binding reagent may also be derived from a molecule known to bind a cell surface receptor. For example, the targeting fragment may be derived from low density lipoproteins, transferrin, EGF, insulin, PDGF, fibrinolytic enzymes, anti-HER2, anti-HER3, anti-HER4, annexins, interleukins, interferons, erythropoietins or colony stimulating factors.

Coupling of the Particle to the Binding Reagent

It is up to a person skilled in the art to employ suitable methods of coupling/grafting for suitably preparing the particles coupled to one or more binding reagents. The amount of binding reagent(s) used for surface functionalization of the luminescent nanoparticles is adjusted having regard to the amount of particles.

Typically, it is desirable for each nanoparticle to be coupled to several binding reagents, preferably at least five binding reagents, and more preferably at least ten binding reagents.

The binding reagent may be grafted directly, or via a spacer (also called “linker”), to the nanoparticle.

The methods of coupling (also called grafting) of the particles to biomolecules are familiar to a person skilled in the art. It is generally coupling by covalent bond, by surface complexation, by electrostatic interactions, by encapsulation, or by adsorption.

In certain cases, including the case of coupling by covalent bond, the particles may be functionalized beforehand with chemical groups that are then capable of reacting with another chemical group carried by the binding reagent to form a covalent bond.

As examples of chemical groups that may be present on the surface of the nanoparticles, we may mention the carboxyl, amino, thiol, aldehyde and epoxy groups.

Amino groups may be supplied by molecules such as the amino organosilanes, such as aminotriethoxysilane (APTES). The advantage of APTES is that it forms, by means of covalent bonds, a capsule around the nanoparticle. The amines supplied by APTES are thus very stable over time. The amino groups may be transformed into carboxyl groups by reaction with succinic anhydride.

Carboxyl groups may be supplied by molecules such as citric acid or a polyacrylic acid (PAA).

The carboxyl groups may be activated by any technique known by a person skilled in the art, in particular by reaction with 1-ethyl-3-(3-dimethylaminopropyl)carbodiimide (EDC) and N-hydroxysuccinimide (NHS), then reacting with the amine functions on the surface of a polypeptide and forming a covalent amide bond, when the binding reagent is a protein or an antibody.

Functionalization of the nanoparticles with APTES may be done advantageously after coating the nanoparticles with a layer of silica.

In other cases, the particles may be coupled beforehand to molecules able to allow subsequent coupling to a binding reagent.

For example, the particles may be coupled to streptavidin, which allows coupling to a biotinylated targeting agent.

As an example, application No. FR1754416 illustrates the coupling of nanoparticles to biotinylated antibodies, by coupling the nanoparticles coupled to streptavidin, to biotinylated antibodies. It may also be carried out directly by coupling the antibodies on nanoparticles functionalized with APTES as mentioned above (transformation of the amino groups into carboxyl groups, activation of the carboxyl groups and direct reaction with the amino groups on the surface of the antibodies).

Coupling of the binding reagent on the surface of the nanoparticles may also be effected by any other method known by a person skilled in the art.

It may also be effected advantageously by coating the nanoparticles with a layer of silica, followed by a reaction of coating with APTES, the amine functions of which serve to react with a bifunctional spacer agent comprising two NHS functions. Next, the nanoparticles coupled to the bifunctional spacer agents can react with the amine functions on the surface of a protein (antibody, streptavidin, etc.). This type of coupling method is described in particular in the works by Casanova et al. ([38]) and Giaume et al. ([39]).

Migration Agent

Depending on the charge and the nature of their surface, their shape and their size, the luminescent nanoparticles of the invention may also be coated with an agent, called “migration agent” hereinafter, which facilitates their migration within the capillary action test device, for example within the nitrocellulose membrane.

A person skilled in the art is able to functionalize the nanoparticles suitably with one or more migration agents. In particular, it is to be understood that this migration agent must not disturb the coupling of the nanoparticles to the binding reagent as described above, and in particular the latter's ability, in the capillary action test, to bind specifically to the analyte or to the capturing reagent competing with the analyte.

The migration agents may in particular be selected from stealth agents or passivating agents.

Such agents may be for example chains of polyethylene glycol (PEG) or poly(ethylene oxide) (PEO), in particular silanized; PEO-poly(propylene oxide)-PEO; chains of poly(ethylene oxide) grafted with poly(L-lysine) chains (“poly(L-lysine)-grafted-Poly(Ethylene Glycol)” (PLL-g-PEG)); chains of dextran grafted with poly(L-lysine); poly(p-xylylene) (parylene); poloxamers (triblock copolymers whose central part is a propylene oxide block and the ends are polyethylene oxide blocks, for example those marketed by the company BASF under the name Pluronic©); poloxamines; polysorbates and polysaccharides (for example, chitosan, dextran, hyaluronic acid and heparin), the poly(D,L-lactide-co-glycolide) (PLGA), polylactic acids (PLA), polyglutamic acids (PGA), poly(caprolactone) (PCL), N-(2-hydroxypropyl)-methacrylate (HPMA) copolymers and polyamino acids; anionic surfactants; cationic surfactants; nonionic surfactants and zwitterionic detergents.

Preferably, the migration agents are selected from silanized PEG chains, poloxamers and polylactic acids (PLA). These agents may be deposited on the surface of the nanoparticles by any approach known by a person skilled in the art. For example, they may be adsorbed thereon or may be fixed covalently thereon.

The coupling of the nanoparticles with one or more migration agents may be effected at the same time as that of the binding reagent or reagents, for example by selecting a migration agent bearing an amino group when coupling of the binding reagent to the nanoparticles takes place by reaction on the amino groups of the binding reagent. In this case, the amount of migration agents is to be adjusted relative to the amount of binding reagents, so that the nanoparticle comprises a sufficient number both of migration agents and of binding reagents on its surface.

Capillary Action Test Device

As stated above, the photoluminescent nanoparticles as defined above are employed according to the invention as probes in a capillary action test, for example in a “lateral flow” assay.

The term “lateral flow” refers to a liquid flow in which all the dissolved or dispersed compounds are transported by capillarity, preferably at equivalent speeds and a regular flow rate, laterally through a diffusion means.

The method of the invention may be implemented with any conventional capillary action test device, for example known for probes of the gold nanoparticles type. The capillary action test device may in particular assume any configuration; it may thus have a linear, radial, T-shaped, L-shaped, cross-shaped configuration, etc.

Hereinafter, reference is made more particularly to the appended FIGS. 1 to 3 and 5, which relate to a device for lateral flow assay of the migration strip type.

Moreover, the device used according to the invention may be adapted to an assay of the “sandwich” type or, alternatively, to a “competitive” assay, as detailed hereunder.

Typically, a capillary action test device, in particular a device for lateral flow assay according to the invention, as shown in FIG. 1, comprises a means for capillary action in a reference direction (X), in particular a porous solid support (10), comprising:
a zone (1) for deposition of the liquid sample, and optionally of a diluent;
a zone (2), arranged downstream of the deposition zone, called “labeling zone”, loaded with the photoluminescent inorganic nanoparticles according to the invention (probes) coupled to at least one reagent specifically binding the substance to be analyzed;
a reaction zone (3), also called “detection zone”, arranged downstream of the labeling zone (2), in which at least one capturing reagent specific to the substance to be analyzed is immobilized; and
a control zone (4), located downstream of the detection zone (3), in which at least one second capturing reagent specific to the reagent specifically binding the analyte is immobilized.

In a “sandwich” assay, the reagent specifically capturing the analyte from the detection zone and the binding reagent coupled to the probe are selected for binding respectively and specifically to the analyte, for example at two different epitopic sites of the analyte.

In a “competitive” assay, the binding reagent coupled to the probe is identical or similar to the analyte, for binding to the capturing reagent of the detection zone, competing with the analyte.

The migration control zone (4) indicates to the user that at least part of the sample has passed properly through the porous solid support of the assay device.

The device for lateral flow assay generally further comprises an absorbent pad (5), arranged downstream of the reaction zone and of the control zone, one end of which is in fluidic contact with the porous support. The absorbent pad maintains migration by capillarity and receives the excess liquid sample.

The terms “upstream” and “downstream” refer to the direction (X) of capillary flow in the assay, this migration taking place from the deposition zone (1) (at the upstream functional end) to the detection zone (3), and ending at the level of the absorbent pad (5) (at the downstream functional end) when the latter is present.

Each of the different zones of the porous solid support of the device for lateral flow assay is in fluid communication with the adjacent zone or zones.

“Fluidic contact” between two elements is intended to denote, as is usual for devices for capillary action tests, that the two elements are in physical contact, so as to allow migration of a liquid from the first element into the second. Preferably, this contact is provided by having one element overlap the other, as shown schematically in FIG. 1.

“Means for capillary action” means more particularly a porous solid support (10) allowing migration of a liquid by simple capillary action. The porosity of this support allows capillary flow (or lateral migration) of the sample and/or reagents in the liquid or wet state. The porous support may be selected from the supports already used in known lateral flow assay devices. As examples, it may consist of nitrocellulose, polyester, glass fibers, cellulose fibers, polyether sulfone (PES), cellulose ester, PVDF, etc.

The means for capillary action may consist of one or more separate parts, and the different parts of the support may consist of different materials. When the means for capillary action consists of different parts or different materials, these elements are arranged so as to allow continuity of capillary flow in the means for capillary action.

Typically, the means for capillary action consists of a porous solid support elongated in the direction (X) of capillary action.

Advantageously, it is a porous support (10) in the form of a band or strip. In particular, it may be an immunochromatographic strip consisting of several superposed or overlapping membranes.

According to a particular embodiment, the porous support is a nitrocellulose membrane. As examples of nitrocellulose membranes, we may mention the membranes Millipore™ HF240, Millipore™ HF180, Millipore™ HF135, Millipore™ HF120, Millipore™ HF090, Millipore™ HF075, Sartorius™ CN140, Sartorius™ CN150, FF120 HP membranes (GE), FF80HP membranes (GE), AE membranes (GE), Immunopore membranes (GE).

The size of the porous solid support of a device for lateral flow assay may vary. For example, it may be a band with a length from 30 to 200 mm, preferably from 60 to 100 mm, and with a width from 2 to 10 mm, preferably from 4 to 5 mm.

The device for lateral flow assay according to the invention may for example consist of a chromatographic strip fixed on a rigid support (6).

The rigid support (6) may consist of various materials such as board, plastic-coated board, or more preferably plastics. Preferably, the rigid support is made of polystyrene.

Advantageously, a specific material corresponds to each zone of the means for capillary action.

The deposition zone (1) (also known by the name “Sample Pad” in English-language terminology) of the sample may advantageously be formed from an absorbent porous material. In fact, the deposition zone of the means for capillary action is intended to receive a liquid sample, for example be brought into contact with a stream of urine or a blood sample. This material is selected from suitable absorbents known by a person skilled in the art, and already used in conventional lateral flow assays.

The inorganic photoluminescent nanoparticles (7) as described above are employed at the level of the labeling zone (2) (also known by the name “Conjugate Pad” in English-language terminology) of the means for capillary action, as shown in FIGS. 2 and 3.

As stated above, these nanoparticles (7) are coupled to at least one reagent specifically binding the substance to be analyzed.

In the context of a “sandwich” assay, the binding reagent is capable of binding specifically to the analyte during the lateral flow assay. It may for example be a specific antibody of the analyte.

In the context of a conventional “competitive” assay, the binding reagent is capable of binding specifically to the capturing reagent of the detection zone (3), competing with the analyte. The binding reagent may thus be for example the analyte itself or a suitable analog. “Suitable analog” means an analog binding specifically to the reagent specifically capturing the analyte.

The binding reagent, coupled to the luminescent probes, is also capable of binding specifically to the second capturing reagent (9) immobilized in the control zone.

Preferably, as stated above, the luminescent nanoparticles additionally have, on their surface, at least one agent intended to facilitate their migration in the device for lateral flow assay, such as a stealth agent or passivating agent, for example polyethylene glycol. These agents will thus facilitate the migration of the nanoparticles, if applicable, bound via the binding reagent to the analyte, in the porous support, for example in the nitrocellulose membrane, as far as the detection zone (3).

The inorganic photoluminescent nanoparticles coupled to at least one reagent specifically binding the analyte are immobilized in the dry state in the means for capillary action, but they are free to migrate by capillary action when wet.

Thus, during the assay, the sample that migrates by capillary action through the means for capillary action entrains the nanoparticles coupled to the reagent specifically binding the substance to be analyzed.

A first capturing reagent, specific to the substance to be analyzed, is immobilized at the level of the detection zone (3) (also known by the name “Detection Pad” in English-language terminology) of the means for capillary action of the device for lateral flow assay according to the invention. It is selected in a suitable manner for its ability to bind specifically to the analyte.

In the context of a “sandwich” assay (FIG. 2), the capturing reagent of the detection zone may be of the same nature as the binding reagents as described above for coupling to the photoluminescent nanoparticles. It may be for example an antibody (8) having a strong affinity for the substance to be analyzed.

In the context of a “competitive” assay, the capturing reagent is also able to bind to the binding reagent coupled to the luminescent probes (for example identical or similar to the analyte).

The analyte (11) and the capturing reagent (8) typically form a ligand/receptor, antigen/antibody, DNA/RNA, DNA/DNA or DNA/protein pair.

Thus, if the analyte is an antigen or a hapten, the capturing reagent is for example a specific antibody of the analyte or, if the analyte is an antibody, the capturing reagent is the antigen recognized by the antibody or an antibody specifically recognizing the analyte. If the analyte is a nucleic acid, the capturing reagent is for example a complementary DNA probe.

The capturing reagents are deposited and immobilized at the level of the detection zone, in such a way that they are not mobile when wet. This immobilization may be effected by techniques known by a person skilled in the art, for example by electrostatic interactions in the case of membranes of nitrocellulose or of charge-modified nylon or by hydrophobic interactions in the case of membranes of poly(vinylidene fluoride) (PVDF) or of polyethersulfone (PES).

According to a particular embodiment, the detection zone (3) may comprise one or more regions, separated spatially, on the means for capillary action, for example in the form of bands (one or more test line(s) “T”), functionalized with one or more capturing reagents. The use of several “test lines” is particularly interesting in the context of the use of the assay for multiple detection, in other words for the simultaneous detection of several substances in one and the same sample.

The capillary action test device typically comprises a control zone (4), located downstream of the detection zone (3), used for confirming the validity of the assay, in which at least one second capturing agent (9), specific to the binding reagent coupled to the probes, is immobilized.

This second capturing agent is selected appropriately for its ability to bind specifically to the binding reagent conjugated to the probes. It may be for example a secondary antibody or a specific antigen of the antibody used as the reagent specifically binding the analyte, in the context of an assay of the “sandwich” type.

As with the first capturing reagent of the detection zone (3), this second capturing reagent is immobilized at the level of the control zone (4) in such a way that it is not mobile when wet.

The means for capillary action may optionally be fixed to a solid support (6) such as a plate or a cassette, generally made of plastic.

A capillary action test device according to the invention may comprise in particular a case (12) in which the test strip is placed, said case preferably being closed, except at the level of certain openings provided, as shown schematically in FIG. 8. In particular, an opening (14) is provided above the zone for deposition of the sample. Another opening, constituting the reading window (13), may for example be provided at the level of the detection zone (3) and of the optional control zone (4). Alternatively, two windows may be provided, for observing the detection zone and the control zone, respectively.

Alternatively, to make these zones visible, the case may be transparent or may be provided with one or more transparent parts.

Of course, various configurations of the device, known for conventional capillary action test devices, may be employed. For example, the case comprising the test strip may comprise, at the level of an upper face, at least one hollow relief, the base of which rests on the surface of the strip, forming a well or space for depositing the liquid sample.

The procedure for the capillary action test according to the method of the invention comprises more particularly:

(i) applying the liquid sample to be analyzed, and optionally a diluent, at the level of the deposition zone (1) of the capillary action test device;

(ii) incubating the device until the luminescence generated by the photoluminescent nanoparticles is detected in the reaction zone (3) and/or until the luminescence is detected in the migration control zone (4); and

(iii) reading and interpreting the results.

The liquid sample to be analyzed may be deposited directly on the deposition zone (1) of the means for capillary action of the device.

“Liquid sample” means any sample in which the substance to be analyzed is in solution or in suspension. This liquid sample may in particular be any biological fluid or body fluid. The liquid sample may also have been obtained from a biological fluid or body fluid. It may also be a liquid extract from a solid sample.

Typically, the liquid sample is urine, whole blood, plasma, serum, diluted fecal matter.

According to a particular embodiment, a diluent is used with the sample to be analyzed, in particular when the liquid sample is plasma, serum, whole blood, nasal or vaginal smear or expectoration for example. The diluent is deposited at the level of the deposition zone of the device.

It may be mixed with the sample to be analyzed, prior to deposition of the sample. Alternatively, the diluent may be deposited before or after the sample. This diluent migrates in the porous support, entraining the sample and the probes coupled to the binding reagent. Typically, this diluent comprises a buffered saline solution. It may also comprise a detergent or any other component necessary for the reaction.

The capillary action test device is then incubated for a sufficient time for migration of the liquid sample by capillary action from the deposition zone to the control zone.

More particularly, a lateral flow assay of the “sandwich” type, shown schematically in FIG. 2, proceeds as follows.

When the porous support is brought into contact with the liquid sample containing the analyte (11), the latter migrates by capillary action in this support as far as the labeling zone where the reagent specifically binding the analyte coupled to the probes (7) is located. The analyte (11) thus binds to the luminescent probes (7) by means of the binding reagent.

If the substance to be analyzed is present, the latter will then be immobilized at the level of the detection zone (3) of the capillary action test device by the first capturing reagent (8) fixed at the level of this zone. This will therefore lead to immobilization of the luminescent probes at the level of the detection zone (3).

Presence or absence of the substance to be analyzed in the sample is thus measured by detecting the luminescent probes at the level of the detection zone (3). More particularly, the luminescence detected at the level of the detection zone increases with, in particular is proportional to, the concentration of the analyte in the sample.

The probes in excess, in other words the nanoparticles coupled to a binding reagent that has not reacted with the analyte, migrate to the control zone. In this control zone, the binding reagent binds to the second capturing agent (9), leading to immobilization of the probes in excess at the level of the control zone (4). The user therefore has at his disposal a positive control allowing the migration of the sample and the reagents in the device to be verified, and therefore verifying proper operation of the test.

According to another variant of the method of the invention, the assay employed is of the “competitive assay” type. In the context of a competitive assay, as shown schematically in FIG. 3-a, in the case of absence of the analyte in the sample, the luminescent probes will be immobilized at the level of the detection zone by binding of their binding reagent to the capturing reagent of the detection zone. However, if the analyte is present, the latter will be fixed, in competition with the binding reagent of the luminescent probes, to the capturing reagent of the detection zone.

Thus, in the context of a competitive assay, the luminescence detected at the level of the detection zone decreases with, in particular is inversely proportional to, the concentration of the analyte in the sample.

According to yet another variant of an assay of the “competitive” type, as shown in FIG. 3-b, the analyte is already immobilized at the level of the capture sites of the detection zone, whereas the binding reagent coupled to the luminescent nanoparticles of the labeling zone is able to bind specifically to the analyte, as in a “sandwich” assay.

If the sample contains the analyte, the latter becomes fixed, as in a sandwich assay, to the luminescent nanoparticles via the binding reagent, and therefore cannot bind at the level of the detection zone. However, the probes coupled to the binding reagent that has not reacted with the analyte may bind to the detection zone via the analyte immobilized at the level of the capturing reagents of the detection zone. Once again, the luminescence signal detected at the level of the detection zone will decrease with, in particular will be inversely proportional to, the concentration of the analyte in the sample.

According to one or other of the aforementioned variants of capillary action test, the results are therefore read by detecting the luminescence generated by the nanoparticles immobilized, at the end of the assay, at the level of the capillary action test device, in particular immobilized at the end of migration of the sample at the level of the detection zone (3) and, optionally, at the level of the control zone (4).

Of course, the invention is not in any way limited to the implementation of a capillary action test device as shown schematically in the appended figures.

Other variants of capillary action test device for implementing the method according to the invention may be envisaged, provided that they are suitable for using, as detection probes, the photoluminescent nanoparticles of the invention. For example, they may be devices for capillary action tests of the “Dipstick Lateral Flow” type or else “Vertical Lateral Flow” type, etc.

According to a variant embodiment, it is possible to detect several substances of the sample with a single assay (so-called “multiplexed” detection).

For example, in the context of a “sandwich” assay, it is possible to immobilize, at the level of the reaction zone (3), at the level of separate regions (for example, several test lines “T”), several capturing reagents specific to each of the substances to be analyzed. In this case, the nanoparticles which serve as detection probe may be coupled to the two or more types of capturing reagents specific to each of the substances to be analyzed. In the presence of the different analytes, the probes will become fixed to each of the separate regions comprising the capturing reagents specific to each analyte. The presence and the value of the luminescence signal on each of the reaction zones will correspond to the presence and the concentration of the corresponding analyte. It is spatial multiplexing in this case.

Alternatively, it is also possible to employ, at the level of the labeling zone (2), various probes doped with different lanthanide ions emitting at different emission wavelengths, each of the probes being coupled to a binding reagent specific to each of the substances to be analyzed and, at the level of a single reaction zone (3), several capturing reagents specific to each of the substances to be analyzed. In this case it is multiplexing using several emission colors. In this case, excitation of the crystalline matrix in the UV is particularly advantageous as it makes it possible to excite, with the same excitation wavelength, different lanthanide ions that emit at different wavelengths.

The two approaches, spatial multiplexing and multiplexing with several emission colors, may be combined for carrying out, for example, detection of four analytes with two probes with two different emission colors, each coupled to two types of reagents specific to two of the four substances to be analyzed and two reaction zones, each comprising specific binding reagents of two of the four substances to be analyzed. Thus, the signal with the two different colors on the first reaction zone will indicate the presence and the concentration of the first two analytes; the signal with the two different colors on the second reaction zone will indicate the presence and the concentration of the other two analytes.

Detection of the Luminescence

As with the conventional capillary action tests, evaluation of the test (detection and/or quantification) carried out according to the method of the invention is performed by observing the detection zone and, optionally, the control zone.

More precisely, the results of the assay are read by detecting the luminescence generated by the probes immobilized at the level of the detection zone and/or the control zone, preferably at the level of the detection zone and the control zone.

Detecting Device

Observation of the detection and control zones of the capillary action test device according to the invention employs more particularly a step (i) of excitation of the photoluminescent nanoparticles and a step (ii) of detection of the luminescence emission.

According to another of its aspects, the invention relates to an in vitro diagnostic kit comprising at least:
a capillary action test device according to the invention as defined above; and
a device for detecting the luminescence generated by the probes immobilized at the level of the detection zone and, optionally, of the control zone of the device.

A simple detection setup, comprising an illumination device comprising an excitation source, thus allows the presence of the luminescent probes to be observed.

The excitation must be compatible with the absorbance characteristics of the nanoparticles. The excitation may take place in the UV, in the visible or in the near infrared.

It may be performed using a noncoherent excitation source such as a lamp, a light-emitting diode or a laser.

The excitation source may directly excite the rare earth ions and/or the matrix of the nanoparticles in which the rare earth ions are incorporated. Preferably, the rare earth ions are excited by excitation of the matrix (for example AVO₄, or some other metal oxide matrix) of the photoluminescent nanoparticles immobilized at the level of the detection zone and, optionally, of the control zone, and then subsequent energy transfer from said nanoparticles to the rare earth ions. In the great majority of cases, excitation of the matrix is performed in the UV.

In particular, in the context of nanoparticles of formula A₁₋ₓLnₓVO_{4(1-y)}(PO₄)_{y} (II) as described above, in particular in which y has a value of 0, the luminescence may be detected by excitation of the matrix at a wavelength strictly less than 350 nm, in particular less than or equal to 320 nm, and more particularly less than or equal to 300 nm.

In the case of the AVO₄ matrix, in particular the YVO₄ matrix of the nanoparticles of formula Y₁₋ₓEuₓVO₄, (IV), excitation may be effected at a wavelength between 230 and 320 nm, in particular between 250 and 310 nm and more particularly between 265 and 295 nm. Excitation of the matrix of the nanoparticles is particularly advantageous since the absorption coefficient of the matrix (or the extinction coefficient for nanoparticles in solution) is far greater than that corresponding to direct excitation of the luminescent ions. Moreover, against all expectations, the parasitic background signal generated by excitation in the UV is not of a nature to prevent detection of the signal resulting from the emission of the nanoparticles, even when the analyte is present at low concentration, as illustrated in the examples given hereunder.

In the case of oxide matrixes containing Eu, excitation may be effected at a wavelength between 210 and 310 nm, in particular between 230 and 290 nm and more particularly between 245 and 275 nm.

In this case, excitation may be effected using a UV lamp, a UV light-emitting diode (LED), or a UV laser. The powers and intensities of excitation necessary for detecting the probes can easily be obtained with a UV lamp or a UV LED. Preferably, excitation is effected using an LED, the latter involving little energy loss, and thus little heat to be removed.

Moreover, advantageously, excitation is carried out uniformly on the surface of the strip, in particular at the level of the detection and control zones. This uniformity can be achieved with a UV lamp but also with several LEDs of lower power arranged around the detection and control zones. As an example, as shown in FIG. 7, four groups of four LEDs may be used. The scheme in FIG. 7 indicates one of the possible schemes for arranging several LEDs around the strip's detection and control zones.

The excitation power density may be between 0.5 and 20 mW/cm², in particular between 1 and 10 mW/cm².

In the context of commercial application of a capillary action test according to the invention, assuming that the heat produced during excitation can be removed effectively, a factor of merit can be defined, taking into account the ratio of the sensitivity of detection obtained with a given excitation power to the cost of the excitation source necessary for obtaining the excitation power in question. Advantageously, the diagnostic kit according to the invention makes it possible to optimize this factor of merit.

According to a particularly advantageous embodiment, reading of the results, in particular in the context of qualitative characterization of the analyte, may be carried out by direct naked-eye observation of the capillary action test device, in particular of the detection zone and, optionally, of the control zone, in particular using an emission filter.

The emission filter makes it possible to select the characteristic emission band of the luminescent ions and thus exclude the nonspecific signals. As an example, when Y₁₋ₓEuₓVO₄ nanoparticles are used, the luminous intensity emitted can be detected at the luminescence wavelength of Eu³⁺ in the YVO₄ matrix, namely 617 nm. The emission filter may be an interference filter or a high-pass filter.

Alternatively, the result can be read using simple detection equipment. The latter may comprise an emission filter and a detector.

The detector is a photon detector.

It may be a single detector, in particular of the photomultiplier, photodiode, or avalanche photodiode type, or a detector of the type of an array of photosensitive devices consisting of a 2D surface of detection pixels such as a CCD or EM-CCD camera or a CMOS camera.

Preferably, it is a detecting device, called 2D, such as a camera. It thus makes it possible to obtain a 2D image of the strip used for the lateral flow assay.

For example, it may be the CCD or CMOS sensor of a smartphone.

Advantageously, the capillary action test according to the invention has a low acquisition time of the signal of the emission. In particular, the luminescence can be measured in a few seconds, in particular in less than a second, in particular in less than 100 ms. Preferably, the acquisition time of the signal of the emission must be compatible with the acquisition time of an image with the camera of a smartphone.

Analysis of the Results of the Lateral Flow Assay

The luminescence results can then be interpreted.

Analysis of the results of the lateral flow assay may consist of simple determination of the presence of the probes (qualitative measurement) at the level of the detection zone and/or control zone, for example by simple visual observation with the naked eye or by visual reading of the 2D image of the strip, obtained for example with a CCD camera, for example the photograph recorded by a smartphone.

It makes it possible to conclude whether or not the target substance of the assay is present in the analyzed sample.

For example, in the context of a lateral flow assay of the “sandwich” type, qualitative interpretation of the results may be as follows:
if two bands are present: the test is positive;
if the single control band is present: the test is negative;
if the single test band is present: the test is invalid;
if no band is present: the test is invalid.

Advantageously, the method of detection according to the invention makes qualitative measurement possible up to 10 times, in particular up to 100 times, or even up to 1000 times, lower than the limit of detection of one and the same capillary action test using gold nanoparticles as probes.

Analysis of the results of the capillary action test may also comprise a quantitative characterization of the substance to be analyzed, in other words determination of the concentration of said substance within the sample, by interpreting the luminescence results.

The detection system used according to the invention may then further comprise any means for analyzing the luminescence emission, for example a converter allowing the luminescence signal to be recorded and exploited.

The luminescence measurement can be interpreted by reference to a preestablished standard or calibration.

As seen above, in the context of an assay of the sandwich type, the luminescence signal of the detection zone increases, in particular is proportional to, the concentration of the analyte, whereas it could be inversely proportional in the context of a competitive assay.

Quantification by reference to a calibration may be carried out, for example, using several control bands, called calibration bands, comprising different concentrations of the substance to be analyzed.

Interpretation of the luminescence measurement may in particular exploit the ratio of the luminescence signal from the detection zone to that from the control zone.

These means for interpreting the luminescence may for example be combined within a smartphone application, allowing analysis of the image obtained, and giving a quantitative value of the result obtained.

Alternatively, the detection system used according to the invention may employ a 2D detector, a system for recording the image, and image analysis software.

Alternatively, the 2D detector may be integrated in the reader, and the image recorded may then be transferred to a smartphone or some other system allowing analysis of the image.

Analysis of the results (by means of analysis software, for example), in particular for quantitative characterization of the analyte, may for example comprise determination of the signal corresponding to the detection zone, the control zone and that of the background signal. The value of luminescence of the background signal is subtracted from the value of the other two zones. Then the ratio of the signal from the detection zone to the signal from the control zone is calculated.

More precisely, analysis of the results (by means of analysis software for example or advantageously by means of an application loaded on the detecting device (smartphone or others)), in particular for quantitative characterization of the analyte, may for example comprise (i) determination of the level of luminescence in the detection zone, L_{D}, of the level of luminescence in the control zone, L_{C}, and of the level of luminescence in a zone without the marker L_{B} (background signal) identified by the user, (ii) calculation of the raw signals S_{D} and S_{C} in the form S_{D}/C=(L_{D/C}−L_{B}), (iii) employing an algorithm for maximization of S_{D/C} allowing optimal localization of the detection and control zones, (iv) calculation of the ratiometric signal R=S_{D}/S_{C} or R=S_{D}/(S_{D}+S_{C}) and (v) comparing the value of R against a calibration table for determining the absolute concentration of analyte. The automated positioning of the detection zones (step iii) makes it possible to avoid the bias introduced by the user and obtain a reproducible quantitative measurement. Alternatively, the position of the detection and control bands can be selected completely automatically without the user's intervention. In the latter case, it is important that the positioning of the detection and control bands on the strip and the positioning of the strip in the reader is always identical.

In the context of an assay of the “sandwich” type, analysis of the results preferably comprises calculation of the ratio R=S_{D}/S_{D}+S_{C}. In fact, in the context of an assay of the “sandwich” type, the higher the concentration of the analyte, the larger the signal S_{D} and the smaller the signal S_{C} (fewer probes remain available for migrating to the control zone).

All the elements for excitation, detection of the luminescence and analysis of the results can be combined within a case, called the reader of the capillary action test device, for example a strip reader.

An opening may for example be made in the strip reader for inserting one or more strips for purposes of reading the test result.

An opening containing a USB connection or equivalent may also be provided so as to be able to transfer the recorded images to data analysis equipment.

The method according to the invention advantageously makes it possible to detect a substance of interest in a sample, in a content strictly less than 5 ng/mL, in particular less than 0.5 ng/mL, or even less than 0.05 ng/mL. This performance depends of course on the analyte, as well as on the efficiency of the specific binding reagent used.

Advantageously, the method of detection according to the invention makes quantitative measurement possible up to 10 times, in particular up to 100 times, or even up to 1000 times, below the limit of quantitation of one and the same capillary action test using gold nanoparticles as probes.

The examples and figures presented below are only given for purposes of illustration and do not limit the invention.

### FIGURES

FIG. 1: Schematic representation, in cross-sectional view, of a strip for a lateral flow assay;

FIG. 2: Schematic representation of an assay of the “sandwich” type, before application of a liquid sample to be analyzed comprising the analyte (11) at the level of the deposition zone (1) (top figure) and at the end of the assay (bottom figure);

FIG. 3: Schematic representation of the procedure of a “competitive” assay according to two variants, before application of a liquid sample to be analyzed comprising the analyte (11) at the level of the deposition zone (1) (top figure) and at the end of the assay (bottom figure);

FIG. 4: Images obtained by transmission electron microscopy (TEM) of the nanoparticles obtained according to example 1.1.a. (Scale bar: 60 nm (FIG. 4a) and 5 nm (FIG. 4b), respectively);

FIG. 5: Histogram of nanoparticle size determined from TEM images for a set of about 300 nanoparticles according to example 1.1.a.

FIG. 6: Photographs of strips, according to the assay in example 3, after migration of a solution containing the h-FABP antigen at 5, 0.5, 0.05 ng/mL, illuminated by a UV lamp. The detection band can be seen on the left, and the control band on the right. The absorbent pad can be seen at the right-hand edge of the images. The luminescence signals shown in the photographs were analyzed by ImageJ. The results are shown in FIG. 7.

FIG. 7: Results for the ratio R=S_{D}/S_{D}+S_{C} measured by ImageJ for liquid samples containing 5, 0.5, 0.05 and 0 ng/mL of h-FABP tested with the test strips according to example 3. The points represent the mean value of R and the error bars represent the associated standard deviation for the 3 and 2 strips, respectively;

FIG. 8: Schematic representation in top view of a case comprising a strip for a lateral flow assay;

FIG. 9: Scheme of the strip reader using four groups of four UV LEDs (“LEDs #1” to “LEDs #4”) for excitation of the nanoparticles. The strip may be inserted in the reader at the level of the insertion rail (20). Reading takes place through the opening in the cover, in which a filter is positioned which makes it possible to select the emission of the nanoparticles (centered at 617 nm in the case in the example) and to reject the excitation wavelength (centered at 280 nm in the case in the example). It may be an interference filter or a high-pass filter. A camera, for example the CCD or CMOS camera of a cellphone, is positioned in front of this opening for recording an image.

FIG. 10: Illustration of the analysis of the result for a strip using a dedicated application operating under Android (Samsung). Left: black and white image of the strip with the rectangles, inside which the cumulative levels of luminescence are calculated, from top to bottom, for the detection zone, the zone of the background signal and the control zone. Right: Screenshot of the cellphone on which the Android analysis application is running. We can see the black and white image of the strip with the rectangles, inside which calculation of the cumulative level of luminescence is performed, and the functions “Capture”, “Measure”, “Adjust” and “Save”.

FIG. 11: (A) Absorbance spectrum of a solution of Y_{0.6}Eu_{0.4}VO₄ nanoparticles synthesized according to the example. (B) Emission spectrum of a nitrocellulose membrane glued on a backing card, as used for the lateral flow assays of the example, inserted in a quartz cuvette, excited at 280, 300 and 380 nm (width of the excitation slit: 5 nm). The emission is far more intense after excitation at 380 nm over the whole spectrum and more particularly at 617 nm, the wavelength at which the signal from the probes based on Y_{0.6}Eu_{0.4}VO₄ nanoparticles is detected.

FIG. 12: Excitation spectrum of the Y_{0.6}Eu_{0.4}VO₄ nanoparticles (left-hand part of the figure) with the emission wavelength fixed at 617 nm and emission spectrum (right-hand part of the figure) with the excitation wavelength fixed at 278 nm.

FIG. 13: Excitation spectrum of the YVO₄:Dy 5% nanoparticles (FIG. 13-a) with the emission wavelength fixed at 572 nm and emission spectrum (FIG. 13-b) with the excitation wavelength fixed at 278 nm.

FIG. 14: Excitation spectrum of the YVO₄:Sm 3% nanoparticles (FIG. 14-a) with the emission wavelength fixed at 600 nm and emission spectrum (FIG. 14-b) with the excitation wavelength fixed at 278 nm.

FIG. 15: Excitation spectra of the Y_{0.6}Eu_{0.4}VO₄, Lu_{0.6}Eu_{0.4}VO₄, LuVO₄:Dy 10%, La_{0.6}Eu_{0.4}VO₄ and GdVO₄:Dy 20% nanoparticles, for an emission wavelength fixed at 617 nm for the nanoparticles containing Eu³⁺ ions and at 573 nm for the nanoparticles containing Dy³⁺ ions.

FIG. 16: Emission spectrum of the Lu_{0.6}Eu_{0.4}VO₄ nanoparticles for an excitation wavelength at 278 nm (excitation of the LuVO₄ matrix). The emission has a main peak at 617 nm and two other peaks at 593 and 700 nm.

FIG. 17: Emission spectrum of the LuVO₄:Dy 10% nanoparticles for an excitation wavelength at 278 nm (excitation of the LuVO₄ matrix). The emission has two main peaks at 483 and 573 nm.

FIG. 18: Emission spectrum of the La_{0.6}Eu_{0.4}VO₄ nanoparticles for an excitation wavelength at 278 nm (excitation of the LaVO₄ matrix). The emission has a main peak at 617 and two other peaks at 593 and 700 nm.

FIG. 19: Emission spectrum of the GdVO₄:Dy 20% nanoparticles for an excitation wavelength at 278 nm (excitation of the GdVO₄ matrix). The emission has two main peaks at 483 and 573 nm.

FIG. 20: Absorbance spectra of the Y(VO₄)_{1-y}(PO₄)_{y}:Eu 20% nanoparticles for y=0, y=0.05, y=0.2, y=0.5 and y=1. The initial concentrations before dilution are of the order of 50 mM of vanadate ions.

FIG. 21: Emission spectra of the Y(VO₄)_{1-y}(PO₄)_{y}:Eu 20% nanoparticles for y=0, y=0.05, y=0.2, y=0.5 and y=1 for an excitation wavelength fixed at 278 nm. The initial concentrations before dilution are of the order of 50 mM of vanadate ions.

FIG. 22: Migration of the Lu_{0.6}Eu_{0.4}VO₄—SA and Lu_{0.9}Dy_{0.1}VO₄—SA nanoparticles on “dipstick” strips containing BSA-Biotin immobilized on the control line, in the absence of antigen. The strips are observed under illumination with a UV lamp at 312 nm. Emission detected through an interference filter (Semrock FF01-620/14-25 and FF03-575/25 for the emission of the Eu³⁺ and Dy³⁺ ions, respectively); image taken with an Iphone 6 smartphone. Two clear bands are observed on the control line. The emission of the nanoparticles that have migrated as far as the absorbent pad can be seen on the right-hand side of the image.

### EXAMPLE

1. Preparation of the Photoluminescence Probes

1.1. Synthesis of Photoluminescent Inorganic Nanoparticles

1.1.a Synthesis of Y_{0.6}Eu_{0.4}VO₄ Nanoparticles

Ammonium metavanadate NH₄VO₃ is used as the source of metavanadate ions VO₃⁻, the orthovanadate VO₄³⁻ being obtained in situ following reaction with a base, in this case tetramethylammonium hydroxide, N(CH₃)₄OH. Yttrium nitrate and europium nitrate were used as sources of Y³⁺ and Eu³⁺ ions.

An aqueous solution of 10 mL of NH₄VO₃ at 0.1 M and 0.2 M of N(CH₃)₄OH (solution 1) is freshly prepared.

A volume of 10 mL of another solution (solution 2) of Y(NO₃)₃ and Eu(NO₃)₃ at 0.1 M of ions (Y³⁺+Eu³⁺) is added dropwise by syringe pump to solution 1 at a flow of 1 mL/min.

The molar concentration ratio of Y(NO₃)₃ to Eu(NO₃)₃ is selected as a function of the desired ratio of the Y³⁺ and Eu³⁺ ions in the nanoparticle, typically the molar ratio Y³⁺:Eu³⁺ is 0.6:0.4.

Once the Y(NO₃)₃/Eu(NO₃)₃ solution has been added, the solution becomes diffusive and appears white/milky without formation of precipitate. The synthesis continues until all of the Y(NO₃)₃/Eu(NO₃)₃ solution has been added.

The final solution of 20 mL must now be purified to remove the excess counterions. For this purpose, centrifugations (typically three) at 11000 g (Sigma 3K10, Bioblock Scientific) for 80 minutes, each followed by redispersion by sonication (Bioblock Scientific, Ultrasonic Processor, maximum power of 130 W operating at 50% for 40 s) are used until a conductivity strictly below 100 μS·cm⁻¹ is reached. The conductivity is measured using a chemical conductivity meter.

The synthesis of the Y_{0.6}Eu_{0.4}VO₄ nanoparticles, on the surface of which the tetramethylammonium cations are immobilized, can be described as follows:

NH₄VO₃+2(N(CH₃)₄)OH [symbol] VO₄³⁻+2 N(CH₃)₄⁺NH₄⁺VO₄³⁻+2 N(CH₃)₄⁺NH₄⁺0.6 Y(NO₂)₃+0.4 Eu(NO₃)₃→Y_{0.6}Eu_{0.4}VO₄+2 N(CH₃)₄+NH₄⁺+3NO₃⁻

Visual observation of the solution of nanoparticles, after being left to stand for 16 hours in a bottle, shows a uniformly diffusing solution.

The final solution remains very stable in water, even after several months at the final pH of the synthesis (about pH 5). The solution remains stable including in the synthesis medium (before removing the excess counterions), although of high ionic strength (>0.1 M).

After removal of the counterions, the zeta potential of the nanoparticles, determined with a DLS-Zeta Potential apparatus (Zetasizer Nano ZS90, Malvern), is −38.4 mV at pH 7.

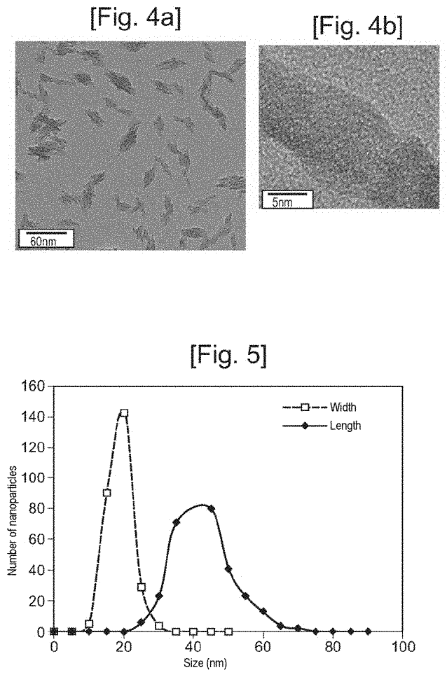

Observation of the nanoparticles by TEM (FIG. 4) shows that the nanoparticles are of elongated ellipsoidal shape. The dimensions of the nanoparticles are determined from TEM images for a set of about 300 nanoparticles (FIG. 5). The nanoparticles of the invention have a length of the major axis, designated a, between 20 and 60 nm, with an average value of about 40 nm, and a length of the minor axis, designated b, between 10 and 30 nm, with an average value of about 20 nm.

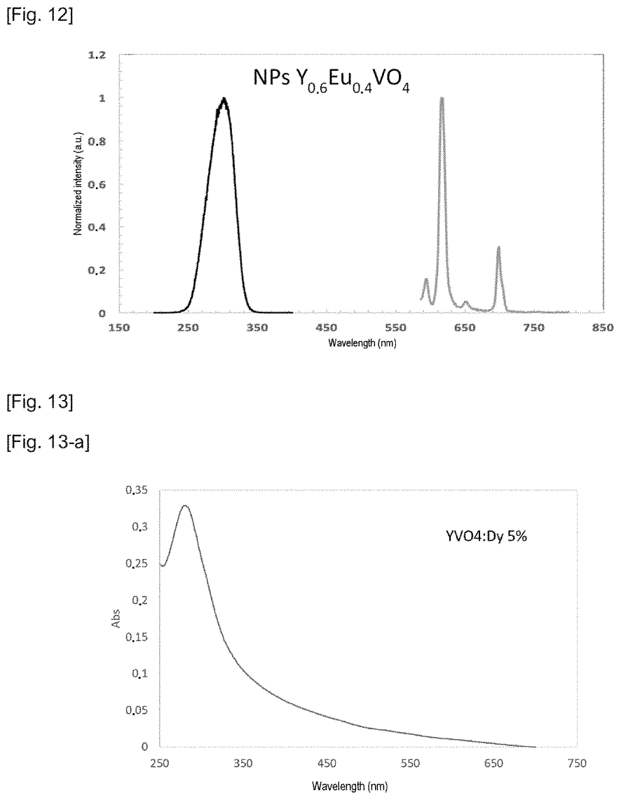

The excitation and emission spectrum of the Y_{0.6}Eu_{0.4}VO₄ nanoparticles is shown in FIG. 12. The excitation spectrum has a peak at 278 nm and the emission spectrum has a main peak at 617 nm and two peaks at 593 and 700 nm.

The Eu³⁺ ions in the YVO₄ matrix can be replaced with other luminescent lanthanide ions. In this case, the excitation and absorption spectrum around the absorption peak of the VO₄³⁻ vanadate ions associated with a V-O charge transfer transition remains unchanged.

The emission spectrum is typical of the emission spectrum of each lanthanide ion.

1.1.b Synthesis of Y_{0.95}Dy_{0.05} VO₄ (YVO₄:Dy 5%) Nanoparticles

The synthesis is identical to that in example 1.1.a., apart from solution 2, which consists of Y(NO₃)₃ and Dy(NO₃)₃ at 0.1 M of ions (Y³⁺+Dy³⁺). Solution 2 is added dropwise by syringe pump to solution 1 at a flow of 1 m/min.

The molar concentration ratio of Y(NO₃)₃ to Dy(NO₃)₃ is selected as a function of the desired ratio of the Y³⁺ and Dy³⁺ ions in the nanoparticle, in this case the molar ratio Y³⁺:Dy³⁺ is 0.95:0.05.

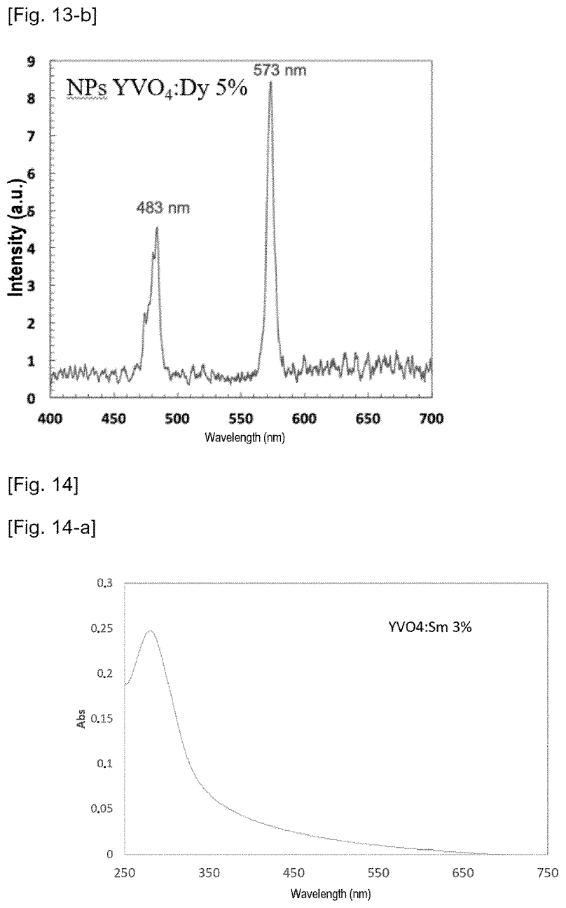

The excitation and emission spectra of these nanoparticles are shown in FIG. 13. The emission of the Dy³⁺ ions has two main peaks at 483 and 573 nm.

1.1.c Synthesis of Y_{0.97}Sm_{0.03}VO₄ (YVO₄:Sm 3%) Nanoparticles

The synthesis is identical to that in example 1.1.a, apart from solution 2, which consists of Y(NO₃)₃ and Sm(NO₃)₃ at 0.1 M of ions (Y³⁺+Sm³⁺). Solution 2 is added dropwise by syringe pump to solution 1 at a flow of 1 m/min.

The molar concentration ratio of Y(NO₃)₃ to Sm(NO₃)₃ is selected as a function of the desired ratio of the Y³⁺ and Sm³⁺ ions in the nanoparticle, in this case the molar ratio Y³⁺:Sm³⁺ is 0.97:0.03.

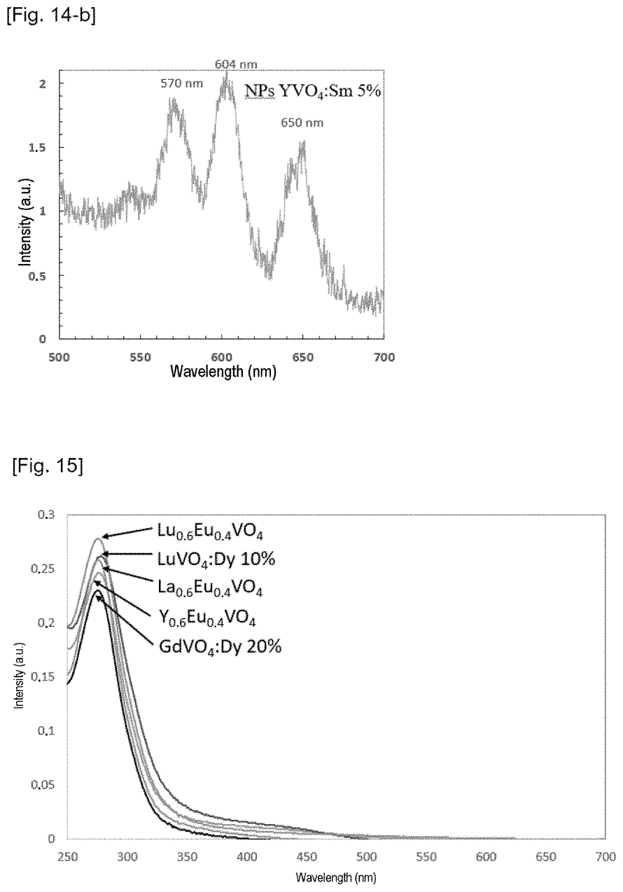

The excitation and emission spectra of these nanoparticles are shown in FIG. 14.

Moreover, the Y³⁺ ions of the YVO₄ matrix can be replaced with other ions such as Gd³⁺, Lu³⁺ and La³⁺ (see next examples). For all these matrixes GdVO₄, LuVO₄ and LaVO₄, the excitation and absorption spectrum around the absorption peak of the VO₄³⁻ vanadate ions associated with a V⁵⁺—O₂⁻ charge transfer transition remains unchanged relative to the YVO₄ matrix. Moreover, in these matrixes GdVO₄, LuVO₄ and LaVO₄, the Eu³⁺ ions can be replaced with other luminescent lanthanide ions. The emission spectrum is typical of the emission spectrum of each lanthanide ion. Different representative combinations of the matrixes and luminescent lanthanide ions are presented hereunder.

1.1.d Synthesis of Lu_{0.6}Eu_{0.4}VO₄ Nanoparticles

The synthesis is identical to that in example 1.1.a, apart from solution 2, which consists of Lu(NO₃)₃ and Eu(NO₃)₃ at 0.1 M of ions (Lu³⁺+Eu³⁺). Solution 2 is added dropwise by syringe pump to solution 1 at a flow of 1 m/min.

The molar concentration ratio of Lu(NO₃)₃ to Eu(NO₃)₃ is selected as a function of the desired ratio of the Lu³⁺ and Eu³⁺ ions in the nanoparticle, in this case the molar ratio Lu³⁺:Eu³⁺ is 0.6:0.4.

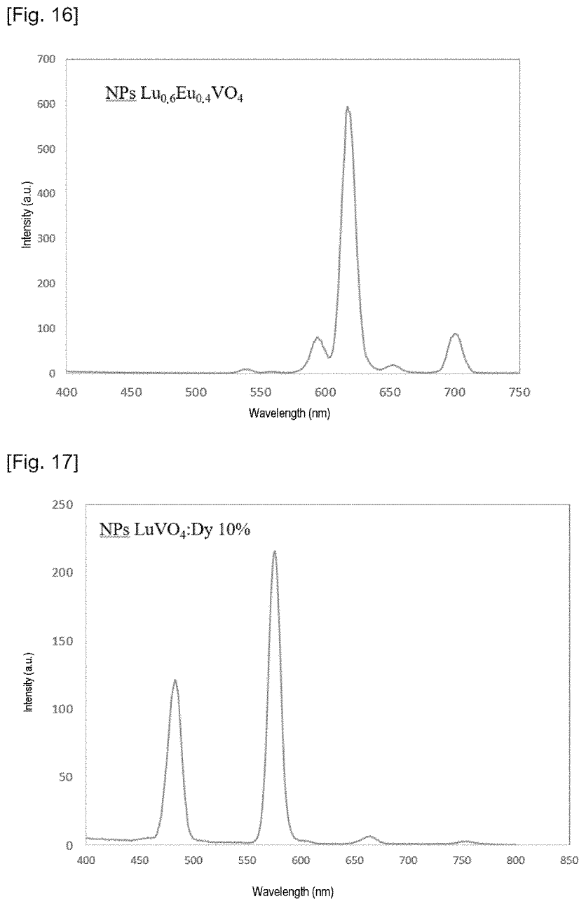

The excitation spectrum of the Lu_{0.6}Eu_{0.4}VO₄ nanoparticles is shown in FIG. 15 and the emission spectrum is presented in FIG. 16. The emission spectrum of the Eu³⁺ ions in the LuVO₄ matrix is practically unchanged relative to that in the YVO₄ matrix (FIG. 12) and has a main peak at 617 nm and two peaks at 593 and 700 nm.

1.1.e Synthesis of LuVO₄:Dy 10% Nanoparticles

The synthesis is identical to that in example 1.1.a, apart from solution 2, which consists of Lu(N03)₃ and Dy(N03)₃ at 0.1 M of ions (Lu³⁺+Dy³⁺). Solution 2 is added dropwise by syringe pump to solution 1 at a flow of 1 m/min.

The molar concentration ratio of Lu(NO₃)₃ to Dy(NO₃)₃ is selected as a function of the desired ratio of the Lu³⁺ and Dy³⁺ ions in the nanoparticle, in this case the molar ratio Lu³⁺ Dy³⁺ is 0.9:0.1.

The excitation spectrum of the LuVO₄:Dy 10% nanoparticles is shown in FIG. 16 and the emission spectrum is presented in FIG. 17. The emission spectrum of the Dy³⁺ ions in the LuVO₄ matrix is practically unchanged relative to that in the YVO₄ matrix (FIG. 13) and has two emission peaks at 483 and 573 nm.

1.1.f Synthesis of La_{0.6}Eu_{0.4}VO₄ Nanoparticles

The synthesis is identical to that in example 1.1.a, apart from solution 2, which consists of La(NO₃)₃ and Eu(NO₃)₃ at 0.1 M of ions (La³⁺+Eu³⁺). Solution 2 is added dropwise by syringe pump to solution 1 at a flow of 1 m/min.

The molar concentration ratio of La(NO₃)₃ to Eu(NO₃)₃ is selected as a function of the desired ratio of the La³⁺ and Eu³⁺ ions in the nanoparticle, in this case the molar ratio La³⁺:Eu³⁺ is 0.6:0.4.

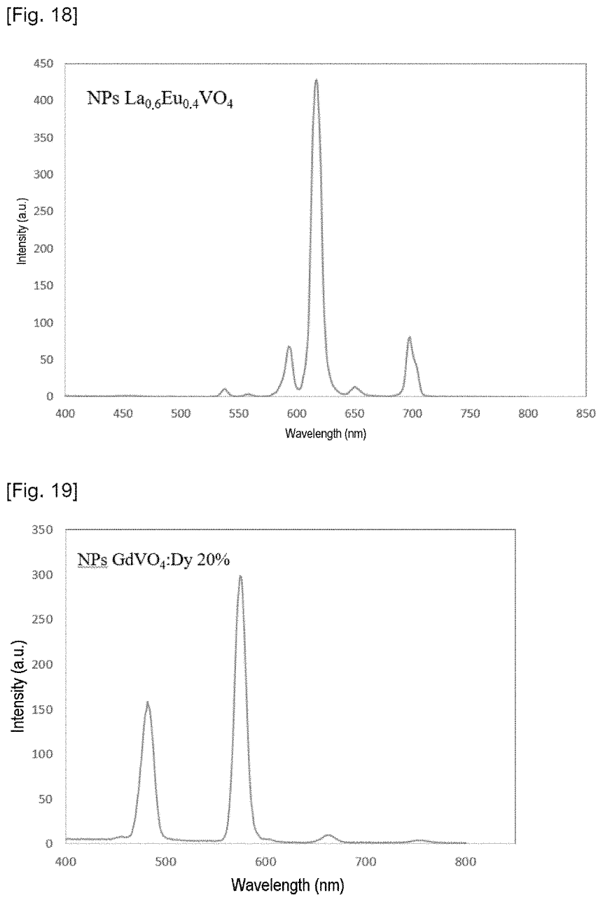

The excitation spectrum of the La_{0.6}Eu_{0.4}VO₄ nanoparticles is shown in FIG. 15 and the emission spectrum is presented in FIG. 18. The emission spectrum of the Eu³⁺ ions in the LaVO₄ matrix is practically unchanged relative to that in the YVO₄ matrix (FIG. 12) and has a main peak at 617 nm and two peaks at 593 and 700 nm.

1.1.g Synthesis of GdVO₄:Dy 20% Nanoparticles

The synthesis of these nanoparticles is carried out starting from an orthovanadate precursor as follows. An aqueous solution of 10 mL of NaVO₄ at 0.1 M (solution 1) is freshly prepared and its pH is adjusted to between 12.6 and 13 with 1 M NaOH solution.

A volume of 10 mL of another solution (solution 2) of Gd(NO₃)₃ and of Dy(NO₃)₃ at 0.1 M of ions (Gd³⁺+Dy³⁺) is added dropwise by syringe pump to solution 1 with stirring, at a flow of 1 mL/min.

The molar concentration ratio of Gd(NO₃)₃ to Dy(NO₃)₃ is selected as a function of the desired ratio of the Gd³⁺ and Dy³⁺ ions in the nanoparticle, in this case the molar ratio Gd³⁺:Dy³⁺ is 0.8:0.2.

Once the Gd(NO₃)₃/Dy(NO₃)₃ solution is added, a milky precipitate forms. The synthesis continues until all of the Y(NO₃)₃/Eu(NO₃)₃ solution has been added. The solution is stirred for 30 min until the pH stabilizes at 8-9.

The final solution of 20 mL must be purified as in example 1.1.a to remove the excess counterions. For this purpose, centrifugations (typically three) at 11000 g (Sigma 3K10, Bioblock Scientific) for 15 minutes, each followed by redispersion by sonication (Bioblock Scientific, Ultrasonic Processor with a maximum power of 130 W operating at 50% for 40 s) are used until a conductivity strictly below 100 μS·cm⁻¹ is reached.

The excitation spectrum of the GdVO₄:Dy 20% nanoparticles is shown in FIG. 15 and the emission spectrum is presented in FIG. 19. The emission spectrum of the Dy³⁺ ions in the GdVO₄ matrix is practically unchanged relative to that in the YVO₄ matrix (FIG. 13) and has two emission peaks at 483 and 573 nm.

1.1.h Synthesis of Y(VO₄)₁₋ᵥ(PO₄)ᵥ:Eu 20% Nanoparticles

Nanoparticles containing a mixture of VO₄³⁻ and PO₄³⁻ ions in the matrix at different VO₄³⁻:PO₄³⁻ ratios were also synthesized.

The synthesis is identical to that in example 1.1.a apart from solution 1, which consists of 0.1·y M of Na₃PO₄, 0.1·(1−y) M NH₄VO₃ at a total concentration of 0.1 M of ions (VO₃⁻+PO₄³⁻) and 0.2·(1−y) M of N(CH₃)₄OH. An aqueous solution of 10 mL with the above concentrations (solution 1) is freshly prepared. NPs with y=0, y=0.05, y=0.2, y=0.5 and y=1 were prepared.

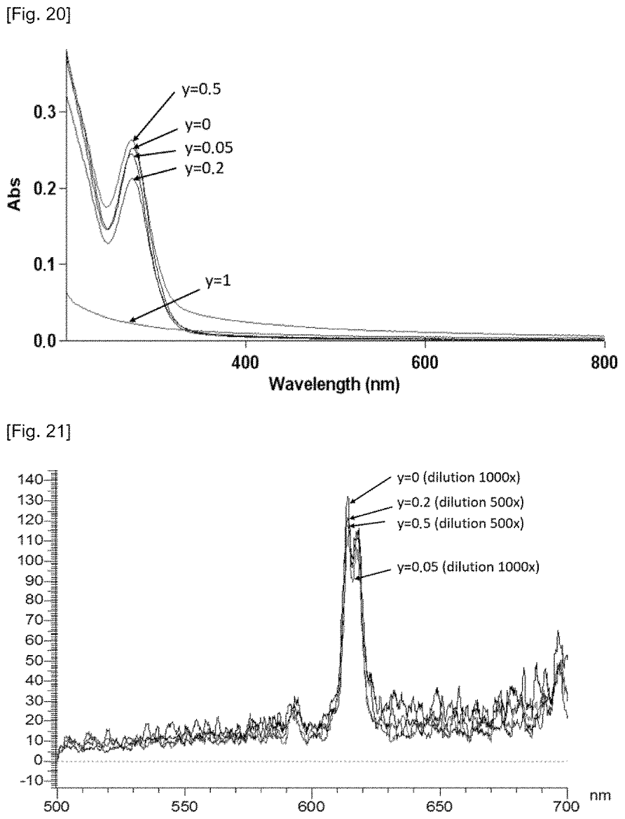

The PO₄³⁻ ions do not display absorption at 278 nm. Thus, the nanoparticles containing 100% of PO₄³⁻ ions do not have an absorption peak at 278 nm (see FIG. 20). The emission spectra of these nanoparticles are presented in FIG. 21 and are identical for all the values of y different from 1 (no emission observed for y=1). They have a main peak at 617 and two additional peaks at 593 and 700 nm. These emission spectra are practically identical.

1.2. Covalent Coupling of the Nanoparticles to Proteins (Anti-h-FABP Antibodies)

The Y_{0.6}Eu_{0.4}VO₄ nanoparticles, obtained as described at point 1.1.a., are coupled to antibodies according to the following protocol.

1.2.1. Coating the Nanoparticles with a Layer of Silica

At the end of synthesis of the nanoparticles, the solution of nanoparticles is centrifuged at 17 000 g for 3 minutes, to precipitate any aggregates of nanoparticles, and the supernatant is recovered. Selection by size is carried out. For this purpose, several centrifugations are carried out at 1900 g for 3 min. Each centrifugation is followed by redispersion of the nanoparticles with the sonicator, and then the size of the nanoparticles is determined using DLS-Zeta Potential apparatus (Zetasizer Nano ZS90, Malvern).

A volume of 25 mL of Y_{0.6}Eu_{0.4}VO₄ particles with a concentration of 20 mM of vanadate ions is prepared. A volume of 2.5 mL of another solution of pure sodium silicate (Merck Millipore 1.05621.2500) is added dropwise by pipette in order to coat the surface of the particles. This solution is left to act with stirring for at least five hours.

The solution is then purified in order to remove the excess silicate and the sodium counterions. The solution is centrifuged at 11000 g (Sigma 3K10, Bioblock Scientific) for 60 minutes and then redispersed by sonication (Bioblock Scientific, Ultrasonic Processor, operating at 50% at a power of 400 W). This step is repeated until the conductivity of the solution is below 100 μS/cm.

1.2.2. Grafting of Amines on the Surface of the Nanoparticles

Put 225 mL of absolute ethanol in a 500-mL flask of the three-necked type, and add 265 μL of APTES (3-aminopropyltriethoxysilane) (Mw 221.37 g/mol Sigma Aldrich), which corresponds to a final concentration of 1.125 mM. This quantity corresponds to 5 equivalents of vanadate that are introduced. A condenser is then attached to the flask. The whole is placed on a flask heater and put under a hood. The mixture is heated under reflux at 90° C. On one of the inlets of the three-necked flask, a colloidal solution of nanoparticles (concentration of vanadate ions [V]=3 mM) in 75 mL of water at pH 9 is added dropwise using a peristaltic pump with a flow rate of 1 mL/min. The whole is heated with stirring for 24 h.

After 48 hours, a rotary evaporator (rotavapor R-100, BUCHI) is used for partially concentrating the nanoparticles. The solution is rotated in a suitable flask, and heated in a bath at 50° C.

The solution recovered is purified by several centrifugations in ethanol:water (3:1) solvent. After purification, sorting by size is carried out following the protocol described above.

1.2.3. Grafting of Carboxyls on the Surface of the Aminated Nanoparticles

Solvent transfer is carried out before beginning the grafting.

The grafting protocol is as follows.

Transfer the aminated NPs from the EtOH:H₂O buffer to DMF or DMSO, performing several centrifugations (13000 g, 90 min). The pellet is redispersed by sonication between each centrifugation (20 s at 75%). Measure and determine the concentration of the NPs.

Recover the NPS in 5 mL of DMF and then add 10% of succinic acid anhydride to a glass beaker (i.e. 0.5 g in 5 mL). Leave to react at least overnight under an inert atmosphere, with stirring.

Wash the carboxylated NPs at least twice by centrifugations (13000 g for 60 min, Legend Micro 17r, Thermo Scientific) in order to remove the DMF and the excess succinic acid anhydride.

Resuspend the carboxylated particles in water or MES buffer at pH6 by sonication (Bioblock Scientific, Ultrasonic processor).

1.2.4. Coupling the Nanoparticles to the Anti-h-FABP Antibodies

Coupling of the nanoparticles surface-grafted with COOH is carried out according to the following protocol:
1. Freshly prepare a mixed solution of EDC/Sulfo-NHS (concentration 500 and 500 mg/mL, respectively) in MES buffer (pH 5-6).
2. Add 90 nM of NPs (in this case it is the concentration of nanoparticles calculated from the concentration of vanadate ions according to the reference Casanova et al. [37]) to 3 mL of solution prepared beforehand, and leave to react for 25 min at room temperature, with stirring.
3. Wash the NPs quickly by at least 2 centrifugations (13000 g for 60 min, Legend Micro 17R, Thermo Scientific) with MilliQ water to remove the excess reagents.
4. Recover the last pellet after sonication in sodium phosphate buffer at pH 7.3. Add the required amount of protein (anti h-FABP Antibody, Ref 4F29, 10 E1, Hytest) as a function of the required ratio (Protein:Nps), typically 2 μM for a ratio of 20:1, and 5 mg/mL of mPEG-silane (MW: 10 kD, Laysan Bio 256-586-9004).
5. Leave this solution to react for between 2 and 4 h at room temperature, with stirring.
6. Add the blocking agent (glycine at 1%) so that it reacts with the free COOH and blocks the residual reaction sites on the surface of the NPs. Leave to react for 30 min.
7. Wash the NPs coupled to the proteins several times by centrifugation using centrifuging filters (Amicon Ultra 0.5 mL, Ref UFC501096, Millipore) with PBS pH 7.2. Transfer the NPs to their storage medium: phosphate buffer+Tween 20 (0.05%)+0.1% glycin+10% glycerol. Take 100 μL for the BCA assays. The rest of the solution is divided into aliquots and frozen at −80° C.

Moreover, all of the nanoparticles synthesized according to examples 1.1.b to 1.1.h can be coupled to antibodies, in the same way as for the Y_{0.6}Eu_{0.4}VO₄ nanoparticles.

1.3. Passive Coupling of the Nanoparticles to Proteins (Anti-h-FABP Antibodies)

Passive coupling of the nanoparticles to antibodies, instead of the covalent coupling in example 1.2, can also be carried out as follows.
Centrifuge a solution of 1 mL of nanoparticles (concentration of 5 mM of vanadate ions) for 15 min at 15 000 g.
Take up the pellet in 800 μL of MilliQ water and then redisperse by sonication (Bioblock Scientific, Ultrasonic Processor, maximum power of 130 W operating at 50% for 40 s).
Add 100 μL of a solution of antibodies at 250 μg/mL in potassium phosphate buffer 2 mM pH 7.4.
Incubate while rotating for 1 hour.
Add 100 μL of potassium phosphate buffer 20 mM pH 7.4/1% BSA.
Centrifuge for 15 min at 15 000 g and remove the supernatant.
Take up the pellet in 1 mL of potassium phosphate buffer 2 mM pH 7.4/0.1% BSA. Redisperse by sonication (Bioblock Scientific, Ultrasonic Processor, maximum power of 130 W operating at 50% for 40 s).
Centrifuge for 15 min at 15 000 g and remove the supernatant.
Take up the pellet in 250 μL of potassium phosphate buffer 2 mM pH 7.4/0.1% BSA. Redisperse by sonication (Bioblock Scientific, Ultrasonic Processor, maximum power of 130 W operating at 50% for 40 s).

2. Preparing the Test on Strips of the “Sandwich” Type

To develop rapid tests for determining the presence of a protein qualitatively or quantitatively, it is necessary to optimize the various parameters and find compromises between the reaction time and the sensitivity of the test.

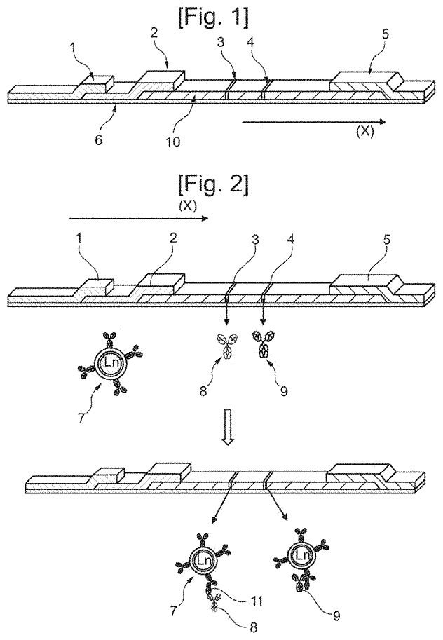

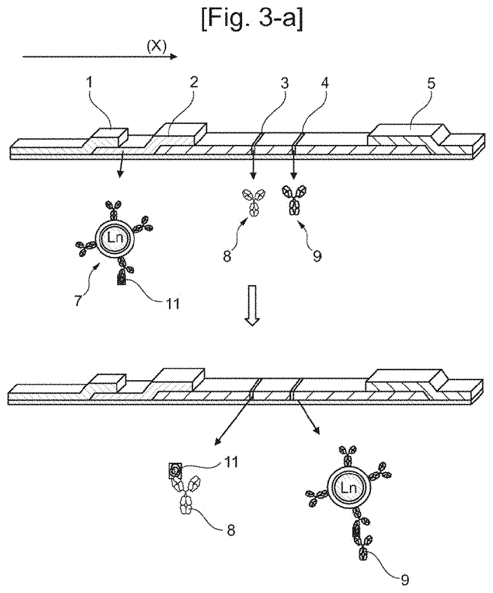

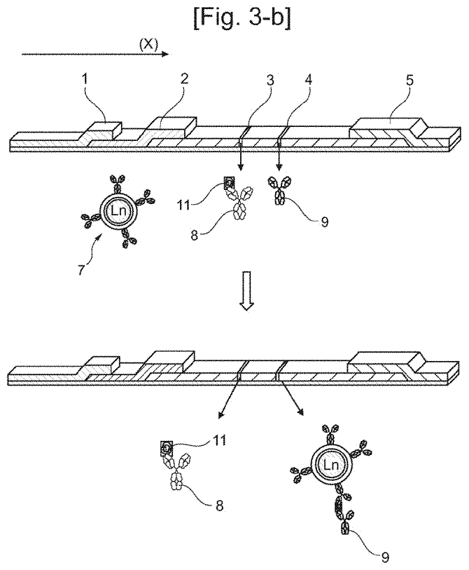

Manufacture of the assay strip, as shown in FIG. 1, is carried out by combining four essential parts:
Essentially inert glass fiber is used as the labeling zone (2) (“Conjugate Pad” in English-language terminology) (GFDX 103000, Millipore).
As deposition zone (“Sample Pad” in English-language terminology) (1) (Ref CFSP173000, Millipore), surface-modified polyesters are used. They have the advantage that they have weak nonspecific interactions with proteins, an excellent traction force as well as good handling properties.
The nitrocellulose membrane (NC) (HF180MC100, Millipore) is used as the means for capillary action (10). It possesses optimal properties for migration of fluids and for immobilization of proteins. The NC membrane is glued on a support (6) of nonporous adhesive plastic (“backing card”).
Cellulose (CFSP173000, Millipore) is used as absorbent pad (5) for its high absorbent capacity.

For detecting h-FABP (human fatty-acid binding protein), which is a cardiac biomarker:

Before assembling the various components, it is necessary to deposit the antibodies on the NC membrane.

1. A solution of mouse monoclonal antibodies directed against h-FABP (ref. 4F29, 9F3, Hytest) is diluted in PBS (pH 7.4) at a concentration of 1 mg/mL. This solution will be used for the test band (3). Another solution of goat polyclonal IgG antibodies (Ref ab6708, Abcam) directed against the mouse antibodies is diluted in PBS (pH7.4) at a concentration of 1 mg/mL. The latter is used for the control band (4).

2. The antibody solutions are deposited on the NC membrane using a “dispenser” (Claremont Bio Automated Lateral Flow Reagent Dispenser (ALFRD)). Using a syringe pump, a volume of 0.7 μL/2 mm is deposited for each band all the way along the NC membrane (about 30 cm long, from which several strips will be made). Leave to dry for 1 h at 37° C.

3. After depositing the antibodies, incubate the NC membrane with 1% BSA diluted in PBS (pH 7.4)+Tween 20 at 0.04% for 30 min at 37° C. to passivate the fixation sites not occupied by antibodies.

4. Deposit the Abs coupled to the NPs, on the labeling zone (“conjugate pad”), using the dispenser. A volume of 3 μL/4 mm is applied all the way along the glass fiber membrane. Leave to dry for 1 h at room temperature before blocking with BSA 1% diluted in PBS (pH7.4). Leave to dry at room temperature.

Assembling the Strip

1. Assemble the various structures (cellulose which serves as absorbent pad and deposition zone, the labeling zone on which Ab+NPs is deposited) on the adhesive parts of the NC on which the Abs are already immobilized. The components are fixed on the plastic support of the NC in the following order: labeling zone (“conjugate pad”), deposition zone (“sample pad”) and finally absorbent pad. For better migration of the fluid by capillarity, the various components are mounted so as to overlap one another as shown in the illustration (FIG. 1).

2. Cut the assembled membrane using a paper cutter into separate pieces 4 mm wide.

3. The strips are then stored in aluminum bags in the presence of a moisture absorber (desiccant) in an atmosphere with a humidity below 30%.

3. Strip Test Procedure

The strip is prepared using Y_{0.6}Eu_{0.4}VO₄ nanoparticles coupled to the antibodies prepared in example 1.2.

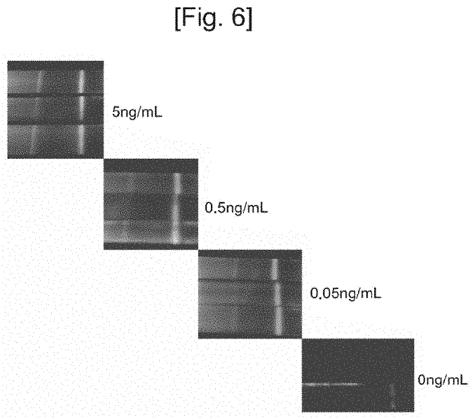

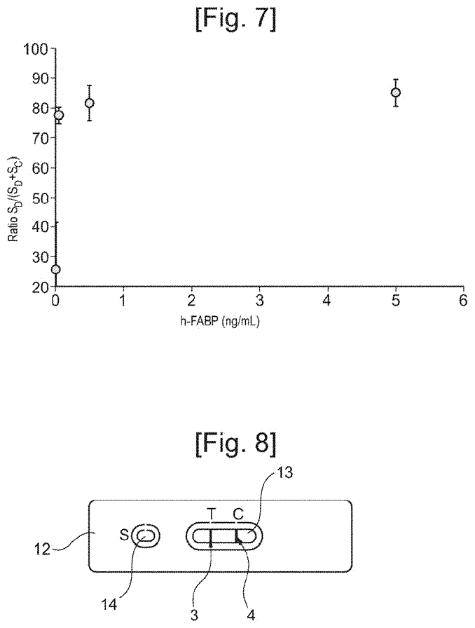

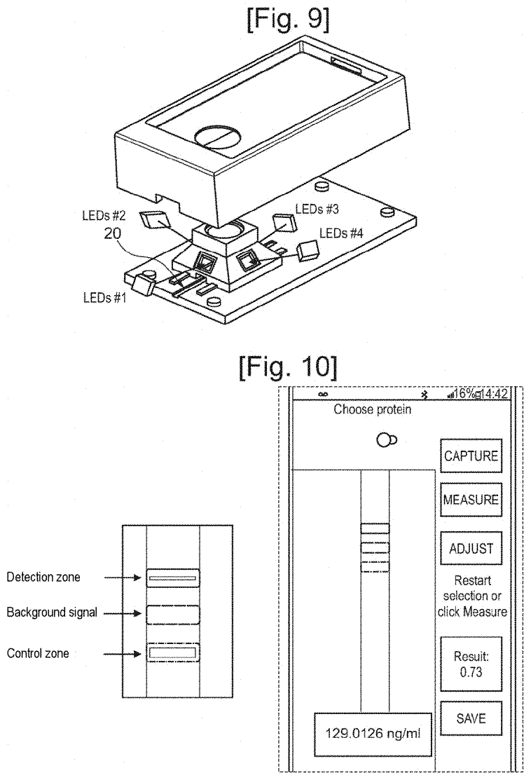

Several concentrations of h-FABP (Ref. 8F65, Hytest) were measured from 5 ng/mL to 0.05 ng/mL. The recombinant h-FABP is diluted to the desired concentrations with buffer or with serum.
1. Bring the strip prepared as described in point 2 above, and the samples to be assayed, back to room temperature before carrying out the test.
2. Deposit 400 μL of the sample in a bottle in the vertical position.
3. Immerse the strip in the bottle, orienting the deposition zone (“sample pad”) downwards. Tap the strip on the bottom to start migration. Keep the strip in the vertical position in the tube for 10 min.
4. Read the results of the strips using a UV lamp (Vilber Lourmat, VL-8.MC 8W at 312 nm and 8 W at 254 nm) (taking digital photographs and analysis by ImageJ, see FIGS. 6 and 7) or using the reader presented in FIGS. 8 and 9. FIG. 10 illustrates analysis of the result for a strip using a dedicated application on a cellphone operating under Android. The reader uses 4 groups of 4 LEDs at 278 nm and an interference filter (620/15, Semrock) for detection. A high-pass filter such as an RG605 filter (Schott) may also be used for detection.

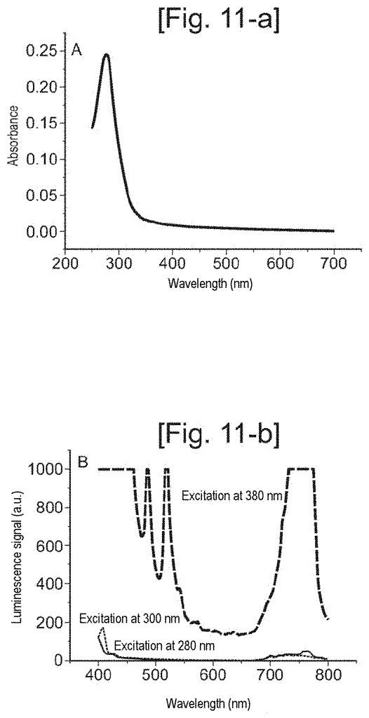

The absorption spectrum of the nanoparticles is presented in FIG. 11 (A). The absorption peak is located at 280 nm with a full width at half maximum of about 50 nm. The emission spectrum of the UV lamp is centered at 310 nm with a full width at half maximum of 40 nm. The emission spectrum of the UV LED is centered at 278 nm with a full width at half maximum of 10 nm.

Qualitative Interpretation of the Results

Qualitative interpretation of the results is as follows:
if two bands are present: the test is positive
if the single control band is present: the test is negative
if the single test band is present: the test is invalid
if no band is present: the test is invalid.

Quantitative Interpretation of the Results

FIG. 10 shows an example of quantitative analysis starting from a digital photograph taken with a cellphone, using a dedicated application. On resting on “Capture” on the phone's screen, recording of a black and white image is triggered. Then, after resting on “Measure”, the application asks the user to point with a finger on the phone's screen to the detection zone and then the control zone so that the application calculates the cumulative luminescence level inside a rectangle containing the detection zone, L_{D}, and the cumulative luminescence level inside a rectangle of the same size containing the control zone, L_{C}. Resting on “Adjust” triggers optimization of the position of the two rectangles corresponding to the detection zone and the control zone so as to maximize the measured signal. The cumulative emission level inside a rectangle of the same size located in the middle of the space between the detection zone and the control zone serves for determining the background signal, L_{B}, and for calculating the signals S_{D/C}=L_{D/C}−L_{B}. The application calculates and then displays the ratio R=S_{D}/S_{C} or R=S_{D}/(S_{D}+S_{C}). The value of R can be compared against a calibration table for also supplying a concentration value in ng/mL. The result can be saved with the “Save” function for later comparison with the next results.

Three strips were prepared for the strip test of each of the samples comprising the h-FABP antigen. For the case of the sample not containing the antigen, only two strips were prepared.

The graph in FIG. 7 shows the results obtained for the ratio R=S_{D}/(S_{D}+S_{C}), measured by ImageJ for the different liquid samples containing 5, 0.5, 0.05 and 0 ng/mL of h-FABP. The points in FIG. 7 represent the mean values of R, and the error bars represent the associated standard deviation for the different strips tested (three strips in the case of the samples containing h-FABP; two strips in the case of the sample not containing it).

Thus, the method according to the invention advantageously allows h-FABP to be detected in a sample, at a content less than or equal to 5 ng/mL, in particular less than or equal to 0.5 ng/mL, or even down to a value as low as 0.05 ng/mL. In other words, h-FABP can be detected at a content less than or equal to 330 pM, in particular less than or equal to 33 pM, or even down to a content as low as 3.3 pM.

4. Migration of the Lu_{0.6}Eu_{0.4}VO₄-SA and Lu_{0.9}Dy_{0.1}VO₄-SA Nanoparticles on “Dipstick” Strips of the “Sandwich” Type

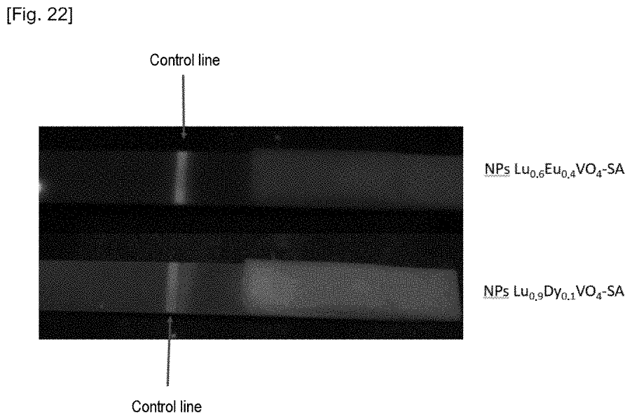

Lu_{0.6}Eu_{0.4}VO₄ and Lu_{0.9}Dy_{0.1}VO₄ nanoparticles synthesized according to examples 1.1.d and 1.1.e, respectively, were coupled to streptavidin (SA) according to example 1.3 (passive coupling). “Dipstick” strips were prepared according to example 2 by immobilizing BSA-Biotin on the nitrocellulose membrane for recognizing the NPs coupled to streptavidin. Test strips were made with the Lu_{0.6}Eu_{0.4}VO₄—SA and Lu_{0.9}Dy_{0.1}VO₄—SA nanoparticles according to example 3, in the absence of antigen. The strips were visualized under UV lamp excitation (312 nm). Two clear, intense bands formed at the level of the control line (FIG. 22).

### REFERENCES

[1] Sajid et al., «Designs, formats and applications of lateral flow assay: A literature review», Journal of Saudi Chemical Society (2015) 19, 689-705;
[2] Kavosi et al., Biosens. Biolectron. 59 (2014) 389-396;
[3] http://www.rapidtest.com/pdf/Troponin_I_Serum%20_WB%20166772-1_%20(05-01-2017).pdf
[4] Huang et al., Biosensors and Bioelectronics 75 (2016) 166-180;
[5] Tang et al., 2009, Biosens. Bioelectron. 25, 514-518;
[6] Xu et al., 2014, Anal. Chem. 86, 7351-7359;
[7] Ji et al. 2015, Talanta 142, 206-2012;
[8] Der-Jiang et al, Journal of Chromatography B, 809 (2004) 37-41;
[9] Khreich et al., Anal. Biochem. 377 (2008) 182-188;
[10] Ahn et al., Clin. Chim. Acta 332 (2003) 51-59;
[11] Biosens. Bioelectron. 63 (2014) 255-261;
[12] Yang et al., Biosens. Bioelectron. 30 (2011), 145-150;
[13] Taranova et al., Biosens. Bioelectron. 63 (2015), 255-261;
[14] Wang et al., Biosens. Bioelectron. 68 (2015), 156-162;
[15] Li et al., ACS Appl. Mater Interfaces 6 (2014), 6406-6414;
[16] Duan et al., Talanta 132 (2015), 126-131;
[17] Ren et al., ACS Appl. Mater Interfaces 6 (2014), 14215-14222;
[18] Medintz et al., Nat. Mat., 2005, 4, pp. 435-446;
[19] Zhang et al., Biosensors and Bioelectronics 51 (2014) 29-35;
[20] Xia et al., Clinical Chemistry 55:1, 179-182 (2009));
[21] Liang et al., Analytica Chimica Acta 891 (2015) 277-283;
[22] Juntunen et al., Analytical Biochem. 428 (2012) 31-38;
[23] Zhiquiang Ye et al., J. Mater. Chem., 2004, 14, 851-856;
[24] DaCosta et al., Anal. Chimica Acta 832 (2014) 1-33;
[25] Achatz et al., Top Curr Chem (2011) 300:29-50;
[26] Lin et al., Biotechnology Advances 30 (2012) 1551-1561;
[27] Dev K. Chatterjee et al., small 2010, 6, No. 24, 2781-2795;
[28] Corstjens et al., Ann. N. Y. Acad. Sci. 1098: 437-445;
[29] Wang et al., Analyst, 2010, 135, 1839-1854;
[30] Niedbala et al., Anal. Biochem. 293, 22-30 (2011);
[31] Maldiney et al., American Chemical Society: Washington, D C, 2012, Vol. 2, pp 1-2;
[32] Paterson et al., Anal. Chem. 2014, 86, 9481-9488);
[33] Min Luo et al., Materials Research Bulletin 48 (2013) 4454-4459;
[34] Takeshitaa et al., Journal of Luminescence 128 (2008), 1515-1522;
[35] Beaurepaire et al., Nano Letters 4, 2079 (2004);
[36] Yuan et al., Trends in Analytical Chemistry, 2006, 2515, pp. 490-500;
[37] Casanova et al. Appl. Phys. Lett., 2006, 89, 253103;
[38] Casanova et al., J. Am. Chem. Soc., 2007, 129, 12592;
[39] Giaume et al., Langmuir, 2008, 24, pp. 11018-11026;
[40] Jan W. Stouwdam et al., Langmuir 2005, 21, 7003-7008;
[41] Wahid et al., J. Solid State Chem. 2016, 245, 89-97 and references cited therein
[42] Riwotzki et al. J. Phys. Chem. B, 1998, 102, 10129;
[43] Meza et al. J. Phys. Chem. A, 2014, 118, 1390;
[44] Dordevic et al. J. Res. Phys., 2013, 37, 47;
[45] Riwotzki et al., J. Phys. Chem. B, 1998, 105, pp. 12709-127;
[46] Huignard et al., Chem. Mater., 2000, 10, pp. 1090-1094;
[47] Huhtinen et al., Anal. Chem. 2005, 77, 2643-2648;
[48] Wolfbeis et al., Angew. Chem. Int. Ed. 2002, 41, No. 23, 4495-4498;
[49] Bouzigues et al., ACS Nano 5, 8488-8505 (2011);
[50] Savin et al., Talanta 178, 910-915 (2018);
[51] Rahman et al., Journal of Colloid and Interface Science 254 (2011), 592-596.

1.-25. (canceled)

26. An in vitro method for detecting and/or quantifying a biological or chemical substance of interest in a liquid sample, by a capillary action test using, as probes, photoluminescent inorganic nanoparticles of the following formula (II):
Al₁₋ₓLnₓVO_{4(1-y)}(PO₄)_{y} (II)
in which:
A is selected from yttrium (Y), gadolinium (Gd), lanthanum (La), lutetium (Lu), and mixtures thereof;
Ln is selected from europium (Eu), dysprosium (Dy), samarium (Sm), neodymium (Nd), erbium (Er), ytterbium (Yb), thulium (Tm), praseodymium (Pr), holmium (Ho) and mixtures thereof;
0<x<1; and
0≤y<1;
said method employing detection of the luminescence, with an emission lifetime shorter than 100 ms, of the nanoparticles, after one-photon absorption, by excitation of the matrix at a wavelength less than or equal to 320 nm.

27. The method as claimed in claim 26, in which detection of the luminescence is effected by excitation of the matrix at a wavelength less than or equal to 300 nm.

28. The method as claimed in claim 26, in which the liquid sample is a biological sample.

29. The method as claimed in claim 26, for detecting and/or quantifying molecules, proteins, nucleic acids, toxins, viruses, bacteria or parasites in a sample.

30. The method as claimed in claim 26, in which said photoluminescent nanoparticles have an average size greater than or equal to 5 nm and strictly less than 1 μm.

31. The method as claimed in claim 26, in which Ln is selected from Eu, Dy, Sm, Yb, Er, Nd and mixtures thereof.

32. The method as claimed in claim 26, in which A is selected from Y, Gd, La and mixtures thereof.

33. The method as claimed in claim 26, in which said nanoparticles have tetraalkylammonium cations on their surface in an amount such that said nanoparticles have a zeta potential, designated ζ, less than or equal to −28 mV, in an aqueous medium of pH≥5, and with ionic conductivity strictly less than 100 μS·cm⁻¹.

34. The method as claimed in claim 26, in which said nanoparticles are of formula A₁₋ₓLnₓVO₄ (III), in which:
A is selected from yttrium (Y), gadolinium (Gd), lanthanum (La), lutetium (Lu), and mixtures thereof;
Ln is selected from europium (Eu), dysprosium (Dy), samarium (Sm), neodymium (Nd), erbium (Er), ytterbium (Yb), thulium (Tm), praseodymium (Pr), holmium (Ho) and mixtures thereof; and
0<x<1.

35. The method as claimed in claim 26, said method using a capillary action test device, in which said photoluminescent inorganic nanoparticles are coupled to at least one reagent specifically binding the substance to be analyzed.

36. The method as claimed in claim 26, said method using a capillary action test device, in which said photoluminescent inorganic nanoparticles are functionalized on the surface with one or more agents intended to facilitate their migration within the capillary action test device.

37. The method as claimed in claim 26, using a capillary action test device, comprising:
a zone (1) for deposition of the liquid sample, and optionally of a diluent;
a zone (2), arranged downstream of the deposition zone, called “labeling zone”, loaded with said photoluminescent inorganic nanoparticles coupled to at least one reagent specifically binding the substance to be analyzed;
a reaction zone (3), also called “detection zone”, arranged downstream of the labeling zone (2), in which at least one capturing reagent specific to the substance to be analyzed is immobilized;
a control zone (4), located downstream of the detection zone, in which at least one second capturing reagent specific to the reagent specifically binding the substance to be analyzed is immobilized; and
optionally, an absorbent pad (5), arranged downstream of the reaction zone and of the control zone.

38. The method as claimed in claim 37, said method comprising at least the following steps:
(i) applying the liquid sample to be analyzed, and optionally a diluent, at the level of the deposition zone (1) of the capillary action test device;
(ii) incubating the device until the luminescence generated by the photoluminescent nanoparticles is detected in the reaction zone (3) and/or until the luminescence is detected in the migration control zone (4); and
(iii) reading and interpreting the results.

39. The method as claimed in claim 26, in which reading of the results of the capillary action test is effected by detecting the luminescence generated by the probes immobilized, at the end of the assay, at the level of the capillary action test device.

40. The method as claimed in claim 26, in which said nanoparticles are of formula Y₁₋ₓEuₓVO₄, (IV), detection of the luminescence being effected by excitation of the YVO₄ matrix at a wavelength between 230 and 320 nm.

41. The method as claimed in claim 26, in which reading of the results of the capillary action test is effected by direct, naked eye observation of the capillary action test device.

42. The method as claimed in claim 26, in which reading of the results of the capillary action test is effected using detection equipment comprising an emission filter and a photon detector.

43. The method as claimed in claim 37, in which interpretation of the results comprises determination of the signal corresponding to the detection zone, the control zone and the background signal of the capillary action test device, subtracting the value of luminescence of the background signal and then determining the ratio of the signal from the detection zone to the signal from the control zone.

44. A capillary action test device, useful for detecting and/or quantifying a biological or chemical substance of interest in a liquid sample, said device comprising, as probes, photoluminescent inorganic nanoparticles of the following formula (II):
A₁₋ₓLnₓVO_{4(1-y)}(PO₄)_{y} (II)
in which:
A is selected from yttrium (Y), gadolinium (Gd), lanthanum (La), lutetium (Lu), and mixtures thereof;
Ln is selected from europium (Eu), dysprosium (Dy), samarium (Sm), neodymium (Nd), erbium (Er), ytterbium (Yb), thulium (Tm), praseodymium (Pr), holmium (Ho) and mixtures thereof;
0<x<1; and
0≤y<1;
said nanoparticles being able to emit luminescence, with an emission lifetime shorter than 100 ms, after one-photon absorption, by excitation of the matrix at a wavelength less than or equal to 320 nm.

45. The device as claimed in claim 44, said device comprising:
a zone (1) for deposition of the liquid sample, and optionally of a diluent;
a zone (2), arranged downstream of the deposition zone, called “labeling zone”, loaded with said photoluminescent inorganic nanoparticles coupled to at least one reagent specifically binding the substance to be analyzed;
a reaction zone (3), also called “detection zone”, arranged downstream of the labeling zone (2), in which at least one capturing reagent specific to the substance to be analyzed is immobilized;
a control zone (4), located downstream of the detection zone, in which at least one second capturing reagent specific to the reagent specifically binding the substance to be analyzed is immobilized; and
optionally, an absorbent pad (5), arranged downstream of the reaction zone and of the control zone.

46. An in vitro diagnostic kit, comprising at least:
a capillary action test device useful for detecting and/or quantifying a biological or chemical substance of interest in a liquid sample, said device comprising, as probes, photoluminescent inorganic nanoparticles of the following formula (II):
A₁₋ₓLnₓVO_{4(1-y)}(PO₄)_{y} (II)
in which:
A is selected from yttrium (Y), gadolinium (Gd), lanthanum (La), lutetium (Lu), and mixtures thereof;
Ln is selected from europium (Eu), dysprosium (Dy), samarium (Sm), neodymium (Nd), erbium (Er), ytterbium (Yb), thulium (Tm), praseodymium (Pr), holmium (Ho) and mixtures thereof;
0<x<1; and
0≤y<1;
said nanoparticles being able to emit luminescence, with an emission lifetime shorter than 100 ms, after one-photon absorption, by excitation of the matrix at a wavelength less than or equal to 320 nm; and
a device for detecting the luminescence generated by the probes immobilized at the level of the capillary action test device, at the end of the assay.

47. An in vitro diagnostic method, said method implementing an in vitro method for detecting and/or quantifying a biological or chemical substance of interest in a liquid sample as claimed in claim 1.

48. An in vitro diagnostic method, said method implementing an in vitro method for detecting and/or quantifying a biological or chemical substance of interest in a capillary action test device as claimed in claim 19.

49. A method for detecting and/or quantifying a substance of interest in an agricultural or food product or in the environment, said method implementing an in vitro method for detecting and/or quantifying a biological or chemical substance of interest in a liquid sample as claimed in claim 1.

50. A method for detecting and/or quantifying a substance of interest in an agricultural or food product or in the environment, said method implementing an in vitro method for detecting and/or quantifying a biological or chemical substance of interest in a capillary action test device as claimed in claim 19.

51. A method for detecting and/or quantifying an illegal chemical substance or any other substance of interest for the police or defense, said method implementing an in vitro method for detecting and/or quantifying a biological or chemical substance of interest in a liquid sample as claimed in claim 1.

52. A method for detecting and/or quantifying an illegal chemical substance or any other substance of interest for the police or defense, said method implementing an in vitro method for detecting and/or quantifying a biological or chemical substance of interest in a capillary action test device as claimed in claim 19.
